(19) United States
(12) Patent Application Publication
(10) Pub. No.: US 2016/0262667 A1
(43) Pub. Date: Sep. 15, 2016

(54) DEVICE FOR MEASURING BIOLOGICAL FLUIDS

(71) Applicant: CoreSyte, Inc., Great Falls, VA (US)

(72) Inventors: Adam Pizer, Jeffersonville, IN (US); Sten Adam Nyberg, Dayton, OH (US); Dalton Pont, Sterling, VA (US)

(73) Assignee: CoreSyte, Inc., Great Falls, VA (US)

(21) Appl. No.: 15/040,319

(22) Filed: Feb. 10, 2016

Related U.S. Application Data
(60) Provisional application No. 62/130,030, filed on Mar. 9, 2015.

(51) Int. Cl. A61B 5/1477; A61B 5/00; A61B 5/145; A61B 10/00

(52) U.S. Cl. CPC A61B 5/1477; A61B 10/0064; A61B 5/4266; A61B 5/14546; A61B 5/14517; A61B 5/443; A61B 5/0004; A61B 5/6833; A61B 5/7475; A61B 2562/164; A61B 2560/0252; A61B 2560/0257; A61B 2503/10

(57) ABSTRACT

A flexible, multi-layered device for automatically sensing sweat biomarkers, storing and transmitting sensed data via wireless network to a computing device having software applications operable thereon for receiving and analyzing the sensed data. The device is functional in extreme conditions, including extremely hot temperatures, extremely cold temperatures, high salinity, high altitude, extreme pHs, and/or extreme pressures.

### CROSS REFERENCE TO RELATED APPLICATIONS

This application claims priority from U.S. Provisional Patent Application No. 62/130,030, filed Mar. 9, 2015, which is incorporated herein by reference in its entirety.

### BACKGROUND OF THE INVENTION

1. Field of the Invention

The present invention is generally directed to fluid sensor devices, and more particularly to a device for sensing and collecting biological fluid inputs.

2. Description of the Prior Art

Generally, biomarkers from biological fluid have significant prognostic and/or diagnostic utility, such as predicting disease, nutritional imbalance, or psychological or physical stress; however, many of the most utilized biomarkers are collected from blood. The ability to predict events through non-invasive means, such as sweat detection, provides great utility to persons under physical stress, particularly individuals in the process of physical activity or exercise. The ability to monitor sweat biomarkers in real time and continuously during activity allows an individual to make informed decisions regarding hydration and exertional status, both variables that moderate physical performance.

For example, hydration status is a predictor of physical performance; dehydration as low as 1% of body mass can impair performance. Prior art detection and treatment, as shown in PRIOR ART FIG. 1, is currently at the stages of when symptoms present, performance degrades, and/or injury presents. Determining hydration through sweat biomarkers before dehydration symptoms present has many benefits, such as reducing fatigue, cramps, and headaches. Therefore, developing a device and system for non-invasively obtaining biomarkers, such as through sweat, is needed.

Sweat contains a multitude of biomarkers; any substance aqueously dissolvable in the blood can present in the sweat by way of eccrine glands. The sweat biomarkers can be small molecules, proteins, metabolites, and/or electrolytes. Well-known electrolytes in sweat are sodium and potassium. As shown in PRIOR ART FIG. 2, potassium concentration is not dependent upon sweat rate due to the passive diffusive transport of potassium, while sodium and chloride concentrations in sweat are dependent upon sweat rate due to the active transport of sodium. Thus, monitoring sodium or chloride concentrations is an accurate, indirect means of indicating hydration status of an individual. Therefore, developing a sweat biomarker device that can communicate to an individual real-time biomarker data is needed.

U.S. Pat. No. 6,198,953 for method and system for continuous sweat collection and analysis by Webster, et al. filed Mar. 11, 1999 and issued Mar. 6, 2001 is directed to a method and system of the invention provide especially for continuously obtaining and analyzing, on a real time basis, sweat from a selected area of skin on the body of a person, especially a neonate, being diagnosed for cystic fibrosis, by causing sweating of the selected area of skin, by placing an electrically positive iontophoretic electrode device of a set of said devices over the selected area of skin preferably within a previously placed receiving and holding device which, following the induction of sweat and removal of the electrically positive iontophoretic electrode device, receives a sweat-sensing electrode device that continuously sends electrical signals to sweat analysis circuitry for providing a digital readout of the ionic composition of the sweat.

U.S. Pat. No. 8,388,534 for an apparatus providing skin care information by measuring skin moisture content by Jang, et al. filed Sep. 24, 2007 and issued Mar. 5, 2013 is directed to an apparatus for providing skin care information, the apparatus including: an electrode unit supplying a voltage to a user's skin and detecting a current signal in the user's skin; a measurement control unit measuring the user's skin moisture content and sweat gland activity by using the detected current signal; a data calculation unit deriving skin moisture content information by using the skin moisture content and the sweat gland activity, and generating skin care information corresponding to the skin moisture content information; and an information provider providing the user with the generated skin care information is provided.

U.S. Pat. No. 7,575,549 for an apparatus and method for increasing, monitoring, measuring, and controlling perspiratory water and solid loss at reduced ambient pressure by Miller filed Jul. 30, 2004 and issued Aug. 18, 2009 is directed to a device for increasing, monitoring, and measuring perspiration water and solid loss at reduced ambient pressure, comprising a sealed chamber capable of maintaining less than atmospheric pressure for an extended period of time and a gasket-sealed door accessing the chamber. An algorithm allowing for continuous calculations of sweat loss and fluid replacement requirements of the occupant of the chamber is disclosed.

US Patent Application Publication No. 2014330096 for performing a physiological analysis with increased reliability by Brunswick filed Nov. 12, 2012 and issued Nov. 6, 2014 is directed to a method for performing an electrophysiological analysis implemented in a system includes: a series of electrodes to be placed on different regions of the human body; a DC voltage source controlled so as to produce DC voltage pulses; a switching circuit for selectively connecting the active electrodes to the voltage source, the active electrodes forming an anode and a cathode, and for connecting at least one other high-impedance passive electrode used to measure the potential reached by the body; and a measuring circuit for reading data representative of the current in the active electrodes, and data representative of the potentials generated on at least certain high-impedance electrodes in response to the application of the pulses, the data allowing a value to be determined for the electrochemical conductance of the skin.

US Patent Application Publication No. 2014350432 for assessment of relative proportions of adrenergic and cholinergic nervous receptors with non-invasive tests by Khalfallah and Brunswick filed Aug. 8, 2014 and issued Nov. 27, 2014 is directed to a system and method for assessing relative proportions of cholinergic and adrenergic nervous receptors in a patient is disclosed. The system includes: an anode, a cathode, and passive electrode for placement on different regions of the patient body. The method generally includes: applying DC voltage pulses of varying voltage values to stress sweat glands of the patient, collecting data representing the current between the anode and the cathode and the potential of the anode, the cathode, and the passive electrode for each of the different DC voltage, and computing data representing the electrochemical skin conductance of the patient. The computed data representing the electromechanical skin conductance of the patient is reconciled with reference data from control patients having known relative proportions of cholinergic and adrenergic nervous receptors. Thus, the relative proportions of cholinergic and adrenergic nervous receptors in the patient can be determined.

US Patent Application Publication No. 2015019135 for motion sensor and analysis by Kacyvensky, et al. filed Jun. 3, 2014 and issued Jan. 15, 2015 is directed to the performance of an individual being monitored based on measurements of a conformal sensor device. An example system includes a communication module to receive data indicative of a measurement of at least one sensor component of the conformal sensor device. The sensor component obtains measurement of acceleration data representative of an acceleration proximate to the portion of the individual. A comparison of a parameter computed based on the sensor component measurement to a preset performance threshold value provides an indication of the performance of the individual.

Published article by Liu, et al. in Conf Proc IEEE Eng Med Biol Soc, 2014:1678-81, discusses the implementation of a microfluidic conductivity sensor: a potential sweat electrolyte sensing system for dehydration detection.

Although biomarkers in sweat are appreciated, specifically electrolytes and glucose, a system and method is still lacking that continuously analyzes sweat biomarkers in real time and transmits data to a user, which informs the user of his or her health status.

### SUMMARY OF THE INVENTION

The present invention is directed to a device for removable attachment to the surface of skin for sensing sweat biomarkers. The device is designed, constructed and configured for sensing and storing biological fluid inputs and for wirelessly transmitting the sensed and stored biological fluid inputs to a computing device with software operable thereon for receiving, storing, and analyzing the biological fluid inputs, calculating biological fluid concentrations and ratios, and providing visual representations of the received and analyzed data on a graphic user interface (GUI) of the computer device.

In a preferred embodiment of the present invention is uniquely configured for active and extended wear in a multitude of climates and weather conditions including wet/submersed use cases and extreme temperatures. The device is also flexible and multi-layered; the layers include the following: a macrofluidic, double-sided adhesive layer; an electronic layer having at least one electrochemical sensor, a microcontroller, and a transceiver antenna coil; a microfluidic management layer; and a vapor porous, top protective layer. The macrofluidic, double-sided adhesive layer is removably adhered to the skin. The electronic layer is intimately adhered to the microfluidic, double-sided adhesive layer. The microfluidic management layer circumferentially surrounds the at least one electrochemical sensor of the electronic layer. The vapor porous, top protective layer is placed on and completely covers the microfluidic management layer and electronic layer. The vapor porous, top protective layer is adhered to the macrofluidic, double-sided adhesive layer. The at least one electrochemical sensor is operable to detect at least one biomarker of a biological fluid, wherein the biological fluid is sweat and the biomarker includes but is not limited to electrolytes, small molecules, proteins, and metabolites.

These and other aspects of the present invention will become apparent to those skilled in the art after a reading of the following description of the preferred embodiment when considered with the drawings, as they support the claimed invention.

### BRIEF DESCRIPTION OF THE DRAWINGS

PRIOR ART FIG. 1 shows a chart demonstrating biomarker types, changes in biomarker levels during progressive stages of injury, and physiological presentations associated with each stage of injury.

PRIOR ART FIG. 2 shows a chart relating sweat rate to concentration of electrolytes.

FIG. 3 shows the multiple layers and associated parts of the sensor apparatus and a top perspective view of the complete sensor apparatus.

FIG. 4 shows a perspective top view of a flexible electronic layer on the bottom adhesive layer.

FIG. 5 shows a complete flexible electronic layer.

FIG. 6 shows a separated layer of electrochemical sensors.

FIG. 7 shows the electrochemical sensors.

FIG. 8 shows a top perspective view (top) and bottom perspective (bottom) view of a complete sensor apparatus.

FIG. 9 shows a diagram of the sweat path through the sensor apparatus.

FIG. 10 shows a diagram of the analytical process within the sensor apparatus.

FIGS. 11A, 11C, and 11D provide tables of various sweat characteristics.

PRIOR ART FIG. 11B shows a chart of sweat characteristics.

FIGS. 12A, 12B, and 12C show electrolyte concentration conversions and ratiometric analysis used within the sensor apparatus.

FIGS. 13A, 13B show thresholds for electrolyte concentrations.

FIG. 14 shows a diagram of the sweat sensor subsystem.

FIG. 15 shows a diagram of components within the sweat sensor subsystem.

FIG. 16 shows a diagram of sweat sensor communications.

FIG. 17 shows a diagram of a NXP semiconductor used in an embodiment of the sensor apparatus.

FIG. 18 shows a diagram illustrating the communication between a NXP semiconductor and a wireless device in an embodiment of the sensor apparatus.

FIG. 19 shows a diagram illustrating a sweat microcircuit of the sensor apparatus.

FIG. 20 shows a diagram illustrating the system architecture.

FIG. 21 shows a diagram illustrating the controls within the user mobile app of the wireless remote transceiver.

FIG. 22 shows a diagram illustrating the network connection between the user mobile app and the user web service.

FIG. 23 shows a diagram illustrating the controls within the cloud database and library, which are part of the user web service.

FIG. 24 shows an individual view of the user web front.

FIG. 25 shows a multi-user view of the user web front.

FIG. 26 shows a diagram of the controls within the present invention's website.

FIG. 27 shows an image of a user database on the remote computer server.

FIG. 28 shows a diagram illustrating the generic cloud architecture.

FIG. 29 shows a diagram illustrating a cloud enterprise.

FIG. 30 shows a diagram illustrating another embodiment of a cloud enterprise.

FIG. 31 shows a diagram of a system for epidemiological research.

FIG. 32 shows a diagram illustrating a manufacturing process for the sensor apparatus.

FIG. 33 shows a schematic diagram illustrating general components of a cloud-based computer system.

FIG. 34 shows a diagram illustrating a suite of sensors in addition to a sweat sensor, including a non-invasive penetration (NIP) sensor.

FIG. 35 shows a top perspective view of one embodiment of the sensor apparatus.

FIG. 36 shows a top perspective view of another embodiment of the sensor apparatus.

### DETAILED DESCRIPTION

The present invention provides an apparatus or device for analyzing biological fluid biomarkers, calculating biomarker data, transmitting data to a transceiver device over a wireless communications network, and storing the data on at least one remote computer and/or server. The present invention further provides a device for removable attachment to the surface of skin for sensing sweat biomarkers under demanding conditions/use cases, wherein the device is designed, constructed and configured for sensing and storing biological fluid inputs and for wirelessly transmitting the sensed and stored biological fluid inputs to a computing device with software operable thereon for receiving, storing, and analyzing the biological fluid inputs, calculating biological fluid concentrations and ratios, and providing visual representations of the received and analyzed data on a graphic user interface (GUI) of the computer device.

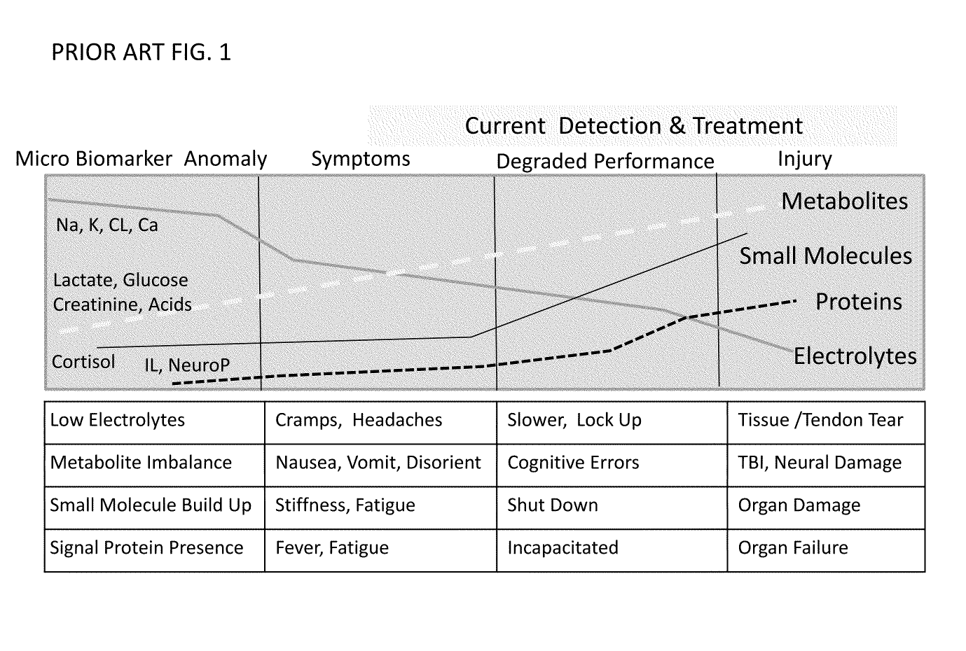

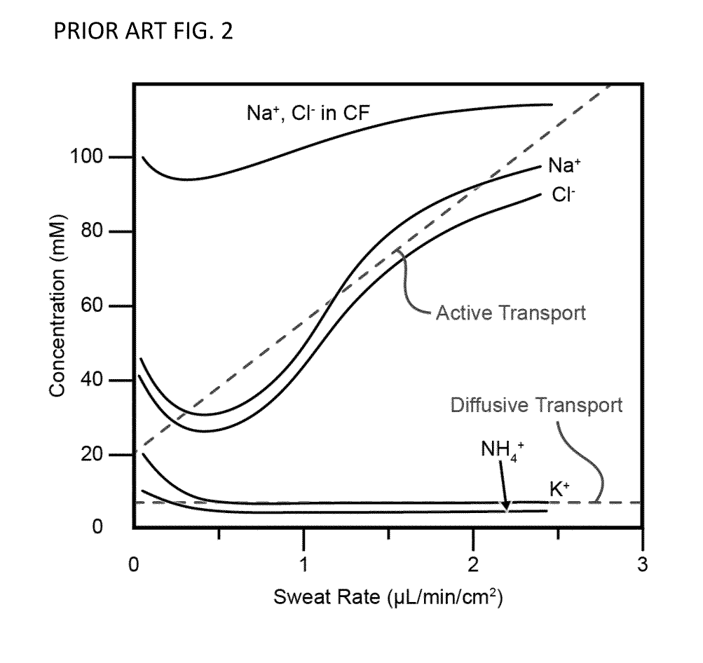

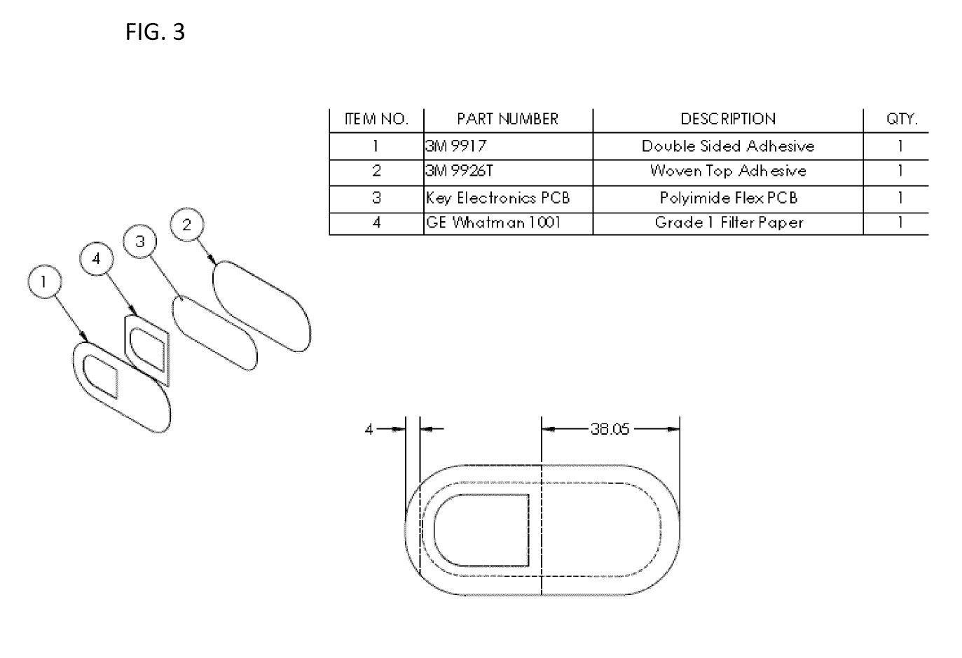

FIG. 3 illustrates a preferred embodiment of the present invention showing a multi-layered apparatus or device that includes a double-sided adhesive layer, a filter paper, a electronic layer, and a woven top adhesive. More specifically, the device is flexible and multi-layered, wherein the layers include the following: a macrofluidic, double-sided adhesive layer; a electronic layer having at least one electrochemical sensor, a microcontroller, and a transceiver antenna coil; a microfluidic management layer; and a vapor porous, top protective layer. The macrofluidic, double-sided adhesive layer is removably adhered to the skin.

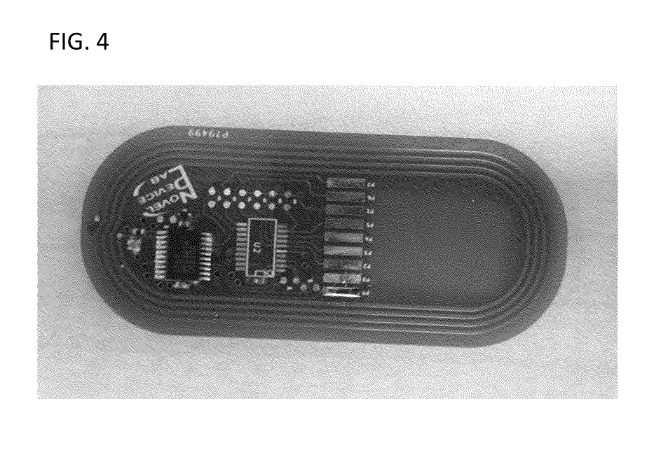

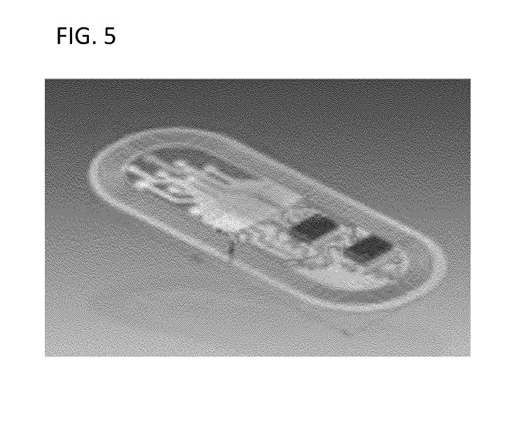

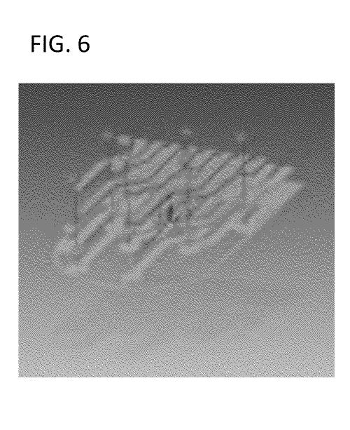

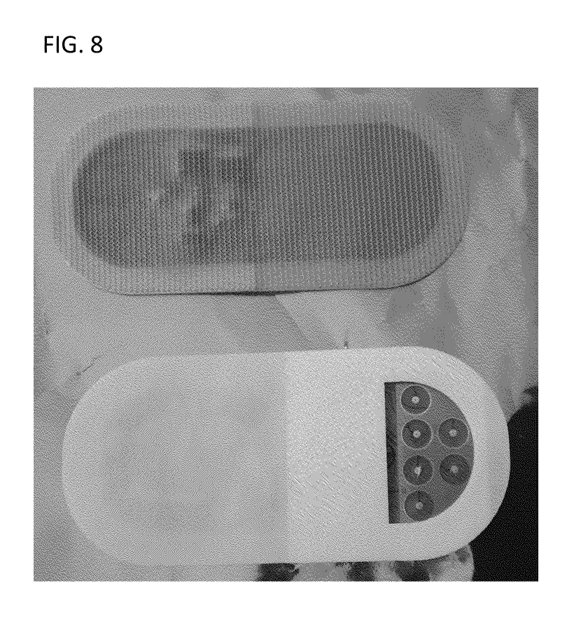

The electronic layer is intimately adhered to the microfluidic, double-sided adhesive layer, as shown in FIG. 4. The microfluidic management layer circumferentially surrounds the at least one electrochemical sensor of the electronic layer. The vapor porous, top protective layer is placed on and completely covers the microfluidic management layer and electronic layer. The vapor porous, top protective layer is intimately adhered to the macrofluidic, double-sided adhesive layer to prevent fluid access to the internal components except through the macrofluidic layer. The fully fabricated sensor apparatus is illustrated in FIG. 8. Preferably, the length of the apparatus is approximately 76.1 mm. In one embodiment, the adhesive of the apparatus is Double Coated Polyester Nonwoven Tape (commercially available as 3M 9917 as of this writing). In another embodiment, the adhesive of the apparatus is Tan Tricot Knit Tape (commercially available as 3M 9917 as of this writing). The design of the microfluidic layer improves flow control and decreases patch layer delamination during high sweat volume use cases.

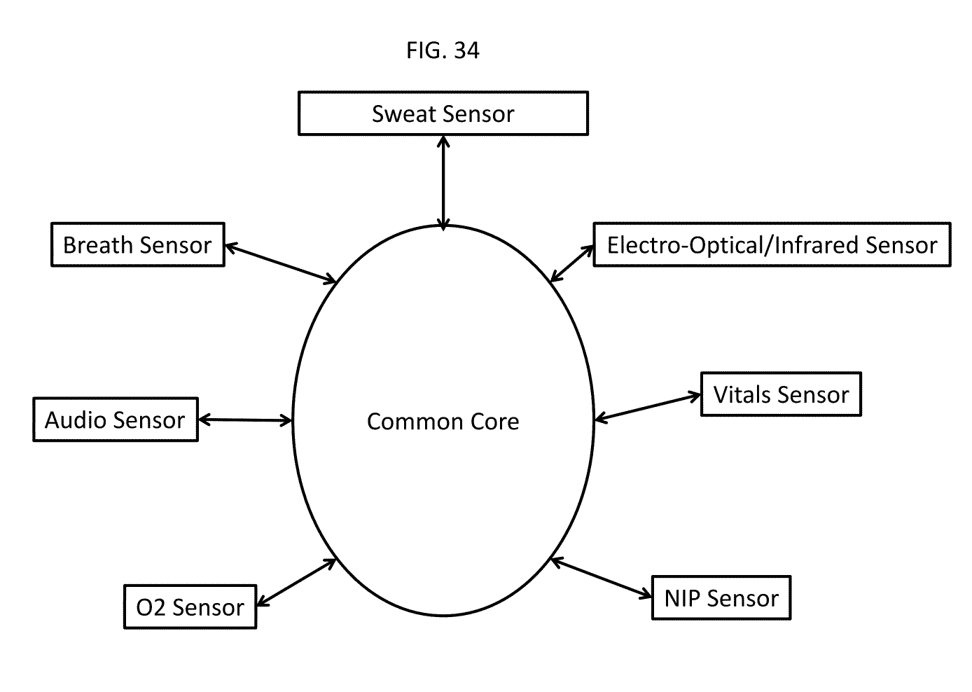

FIG. 34 shows a diagram illustrating a suite of sensors in addition to a sweat sensor, including a non-invasive penetration (NIP) sensor, an audio sensor, a vitals sensor, an electro-optical/infrared sensor, an oxygen sensor, a breath sensor, and a sweat sensor. In one embodiment the sensor includes at least two of the sensors in FIG. 34. In another embodiment, one of the suite of sensors is the only sensor utilized. One or more of the sensors is embedded in the skin in one embodiment of the present invention. An embedded sensor preferably mimics the composition and behavior of cells. The electro-optical/infrared sensor may include a fluorescent signal sensor. In one embodiment, a reader sends an excitation light through the skin to the biosensor, which then emits a fluorescent light proportional to the amount of biochemical measured.

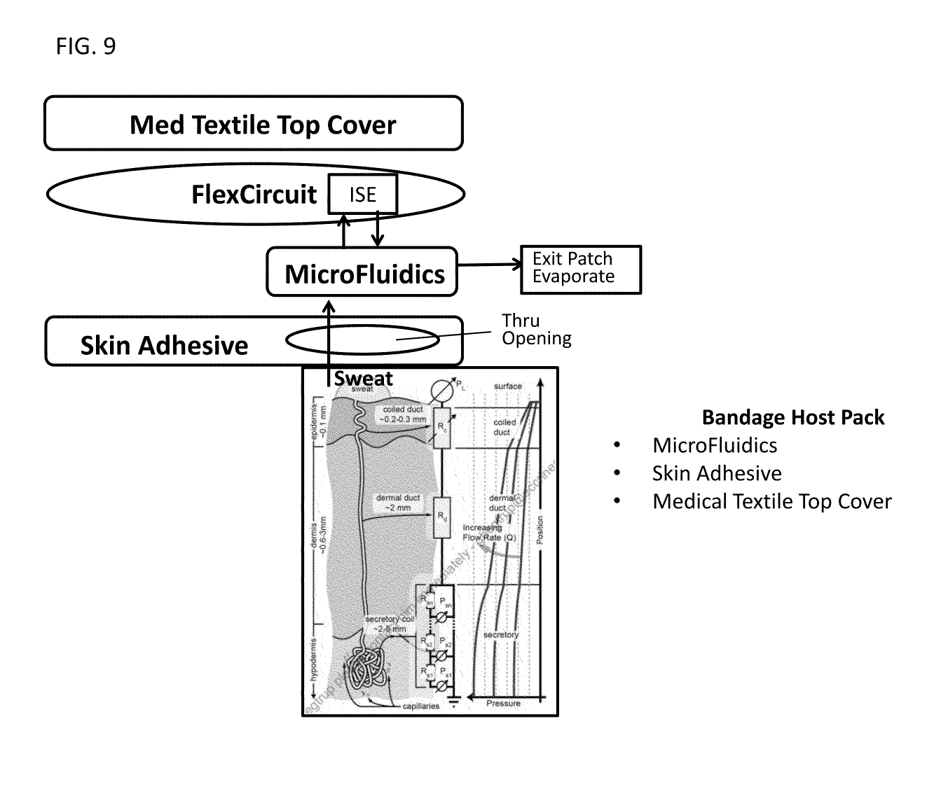

The sensor apparatus is designed to allow sweat to flow through laser cut, macrofluidic pores in the skin adhesive layer, as shown in FIG. 9. Sweat then flows through a filter to the electronics layer, specifically the electrochemical sensor unit, where biomarkers may contact the electrodes of the electrochemical sensor unit. The sweat evaporates through the woven textile protective top layer. The evaporation affords improved and continuous sweat flow into the sensor apparatus. This wicking ensures sweat sensing measures are consistently using new sweat samples rather than static or diluted samples. In one embodiment, the wicking and sweat flow rates range from 0% to 5% Total Body Loss/hr (instant equivalent sweat loss rate) or equivalent to 11.5 L/hr total loss.

The present invention further includes a device with a small amount of ionophore polymer on the active electrodes to filter/prevent untargeted ions to reach the electrode. Sensor functionality and accuracy require precision placement with proper thickness of a small amount of ionophore polymer in one embodiment. The amount is approximately 2 microliters with a designated viscosity placed in a clean assembly environment to completely cover the exposed active electrode on the skin-facing side of the flexcircuit. The coating shall preferably not exceed more than 0.5 mm from the edge of the electrode. In one embodiment, the ionophore polymer is cured. In another embodiment, the curing takes place using heat and/or light to accelerate drying without changing the ionophore selectivity characteristics.

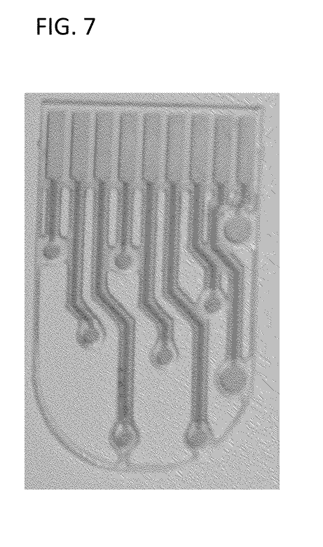

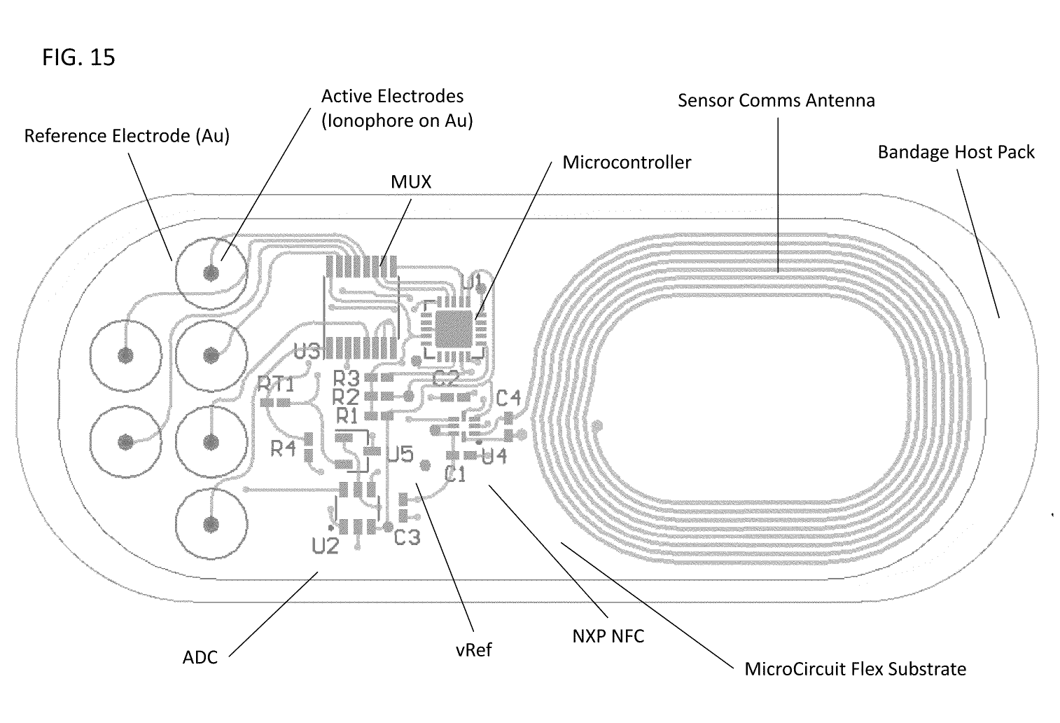

In one embodiment, the sensor is calibrated. Preferably, a user calibrates the sensor using actual test results and feedback from the sensor. The sensor apparatus includes sweat sensor subsystem, as shown in FIG. 15, which includes a microcontroller that receives multiple input data, which are input from multiple sources. A first source is biological fluid, preferably sweat, although alternative fluids may be used. The sweat contains a variety of analytes, such as, by way of example and not limitation, electrolytes, small molecules, proteins, and metabolites. Exemplary analytes include substances including sodium or potassium. In one embodiment, the sensor apparatus is operable to sense sodium and chloride in a dynamic range from about 0 mM to about 120 mM, with normal ranges in humans being about 20 mM to about 100 mM. In another embodiment, the sensor apparatus is operable to sense potassium in a dynamic range from about 0 mM to about 40 mM, with normal ranges in humans being about 5 mM to about 20 mM. In one embodiment, the sensor has a response time of about 60 seconds with about a 90% response. Other analytes include oxygen, glucose, ammonium, and interleukins. In one embodiment, the sensor is operable to analyze the analytes at a pM level, preferably in the 1-10 pM range or even below 1 pM (the sub-pM level). These analytes are collected at the electrochemical sensor, as shown in FIG. 7, which houses reference (preferably standard) and active electrodes. In one embodiment, the apparatus has an embedded dot-circle configuration for a reference electrode to improve stability through less interference. Additionally, gold probes or electrodes are used in one embodiment to improve stability and reduce production costs. The apparatus also includes a microprocessor, multiplexer (mux), ADC, and optimized on board processing for real time, pre-transmission sensor signal conditioning in another embodiment. The apparatus is also optimized for efficient and successful transmission during athletic usage. When the electrodes contact the sweat biomarkers a voltage is produced. Three electrodes per analyte are used to create an average voltage value, which is transmitted to the microcontroller, wherein the microcontroller pre-processes and prepares the sensor data to be communicated wirelessly to the transceiver device via Bluetooth communication in preferred embodiments. Alternatively, a NFC chip or an RFID chip may be provided for communicating sensor data wirelessly. The NFC chip preferably has an increased base signal amplitude for better processing and lower resolution as well as better concentration confidence and resolution. Bluetooth is preferred due to its low energy, ubiquitousness, and low cost. Most any Bluetooth enabled device can pair with another Bluetooth device within a given proximity, which affords more ubiquitous communication between the microcontroller and a transceiver device. The dynamic and automatic connections of Bluetooth allow for multiple microcontrollers to communicate with a single transceiver device, which, by way of example and not limitation, would provide for a team-based situation, wherein the sweat data of multiple athletes is communicated to a single coach or team database.

In one embodiment, the microcontroller receives user data to customize rates at which the at least one biological fluid is sensed and analyzed, to toggle circuit power of the microcontroller, communicate via multiple channels and/or buses with a computing device biological fluid data via multiple protocols concurrently in real-time to a computing device with software operable thereon for receiving, storing, and analyzing the biological fluid data, and define data collection along with transmission characteristics in real time for optimization of power and the electrochemical sensor features for a user, wherein the user data includes heigh, weight, gender, fitness level, conditioning level, age, transepidermal water loss, metabolic disorder, environmental temperature, environmental pressure, environmental sodium concentration, environmental chlorine concentration, and/or combinations thereof

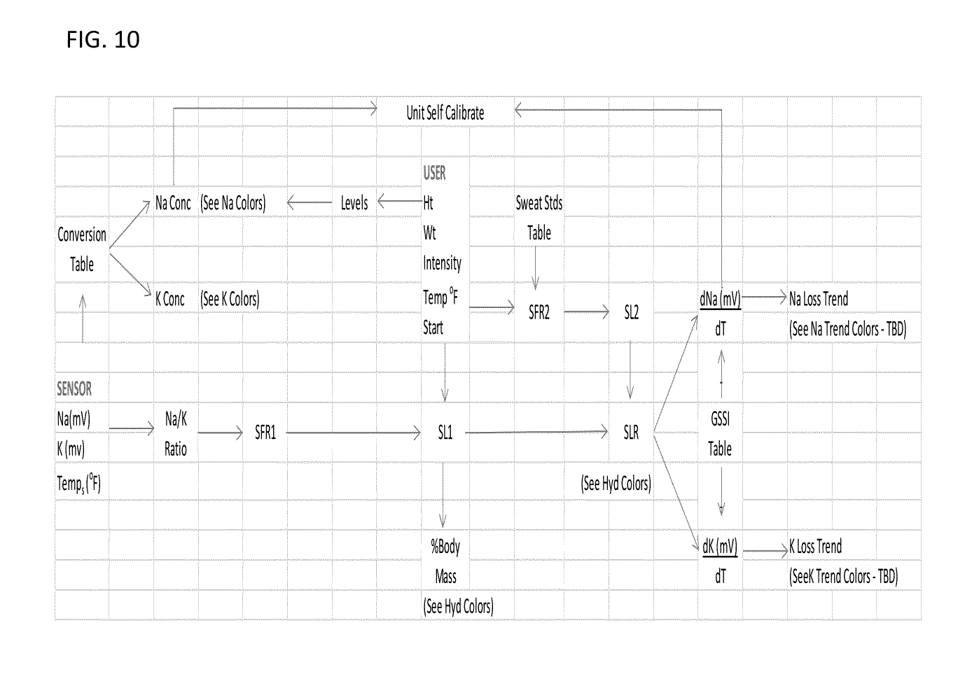

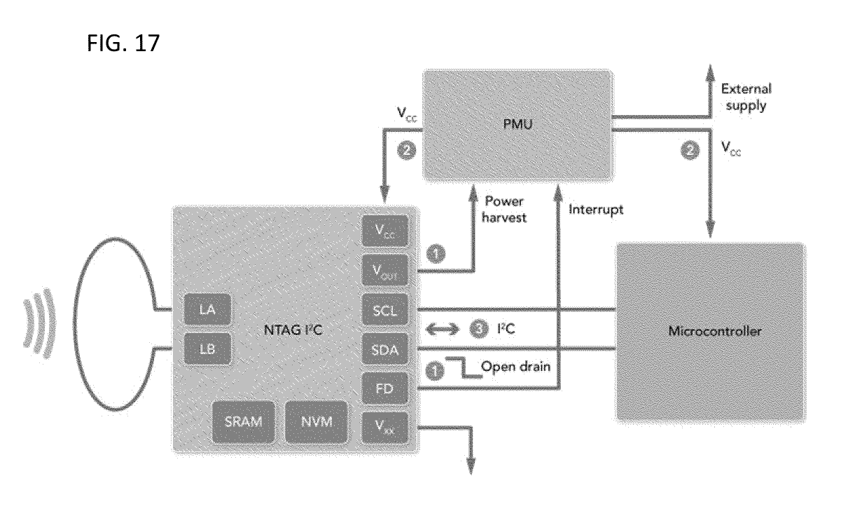

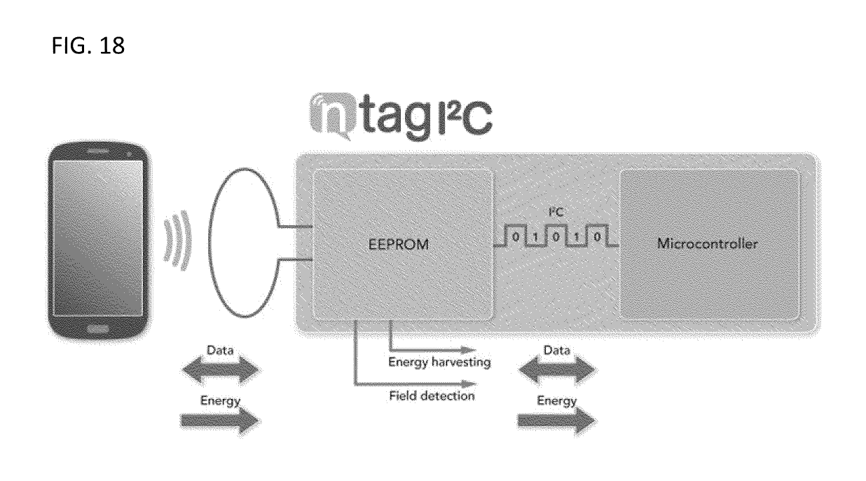

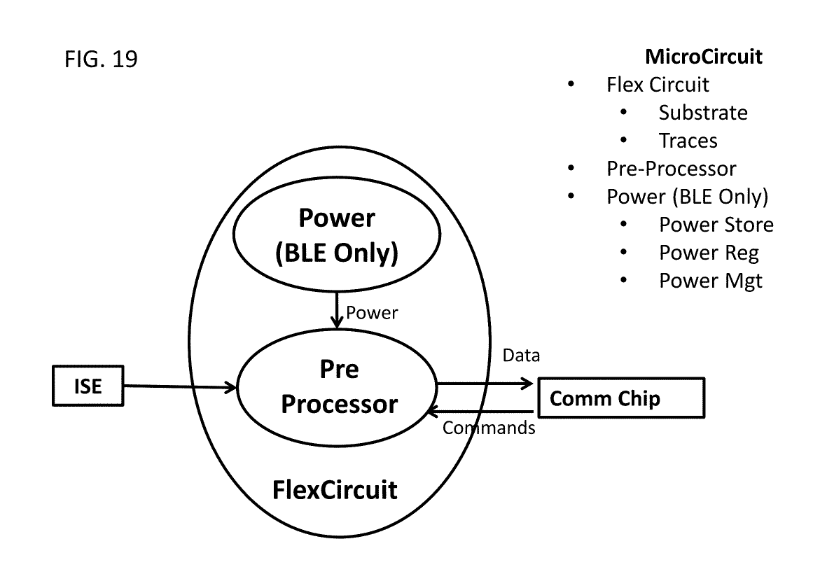

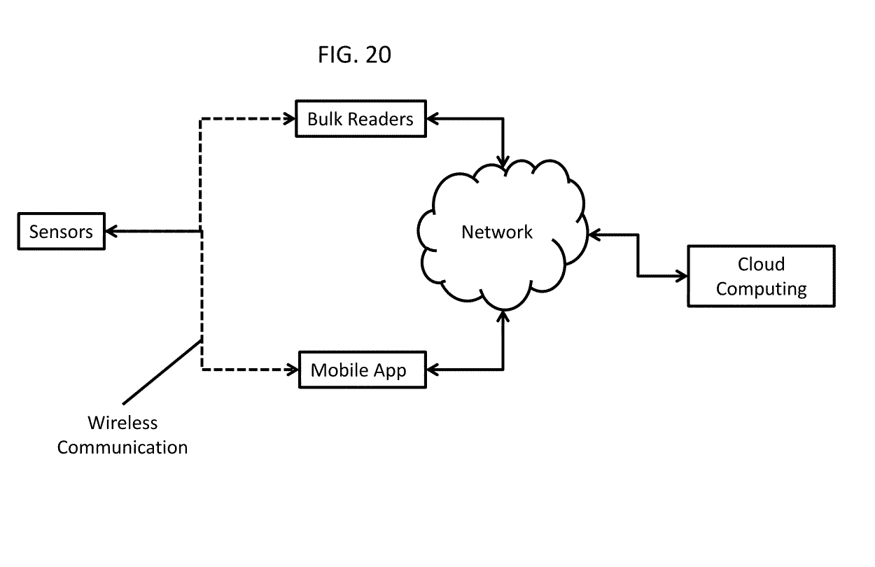

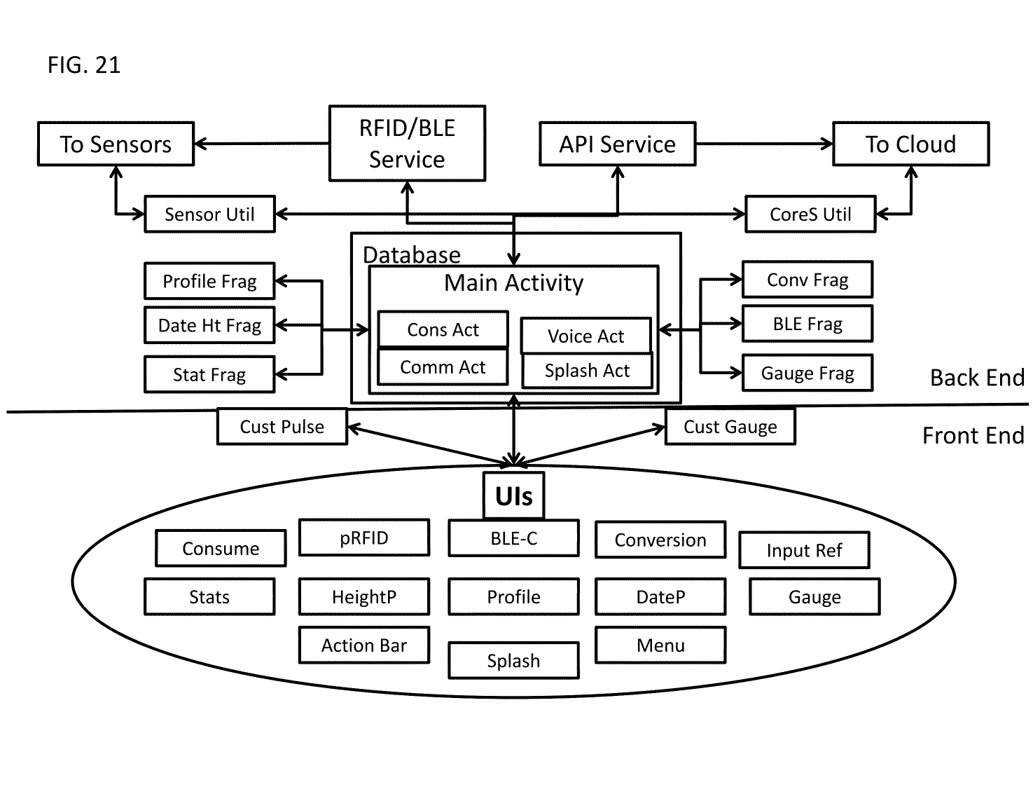

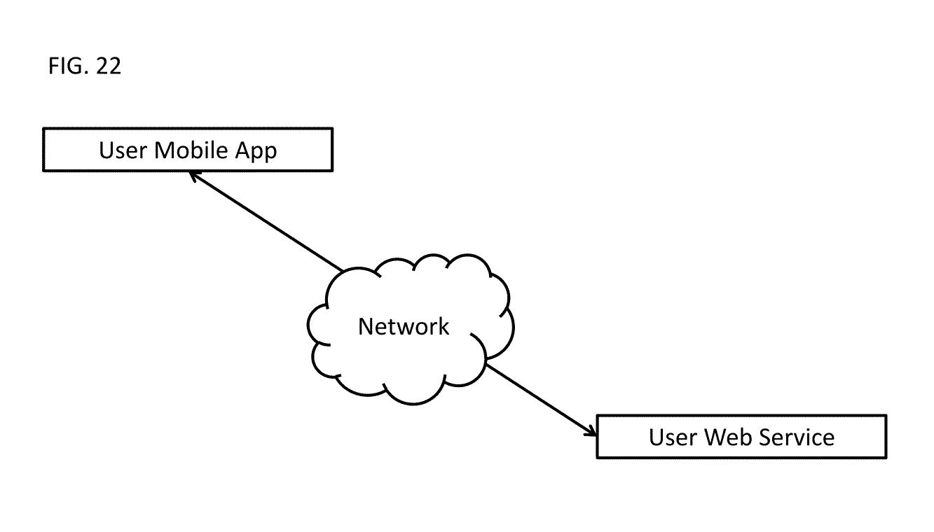

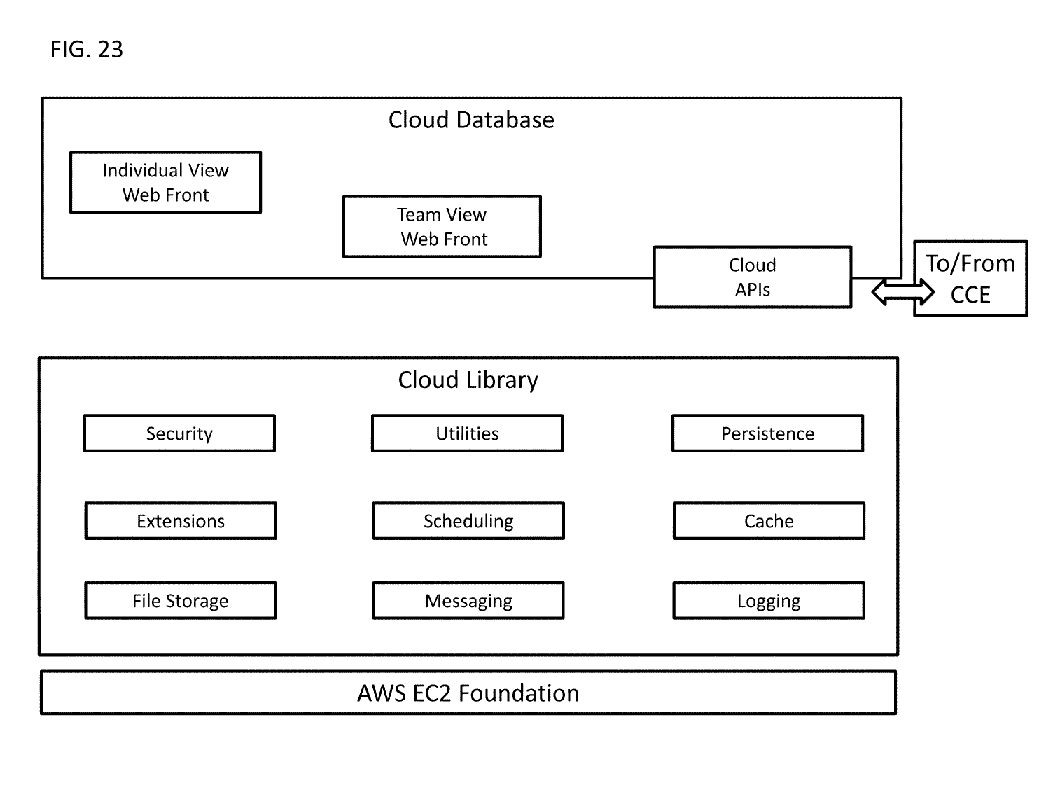

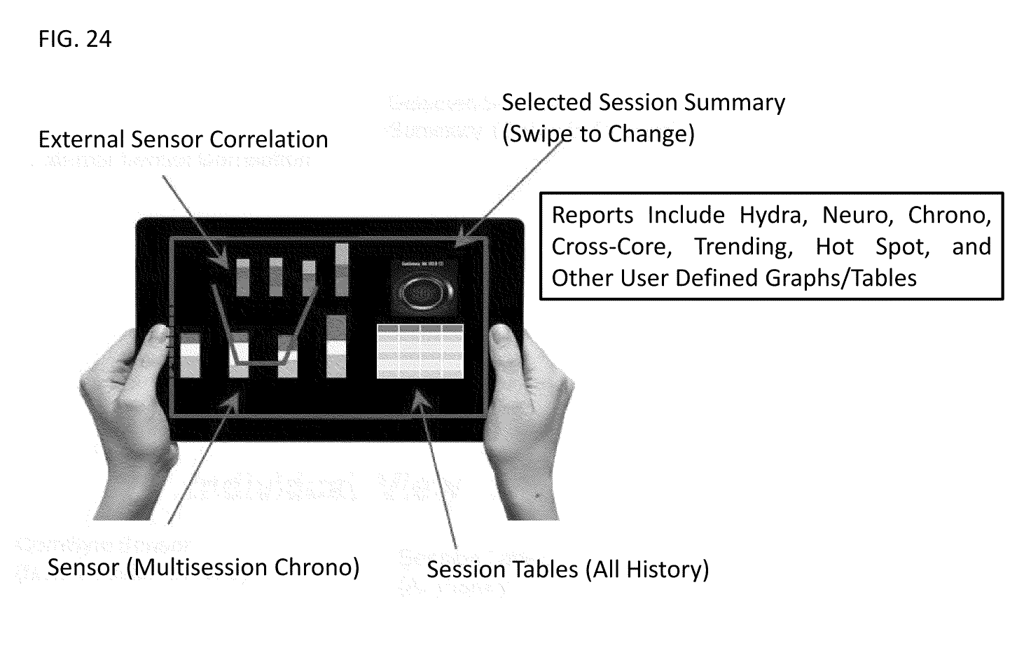

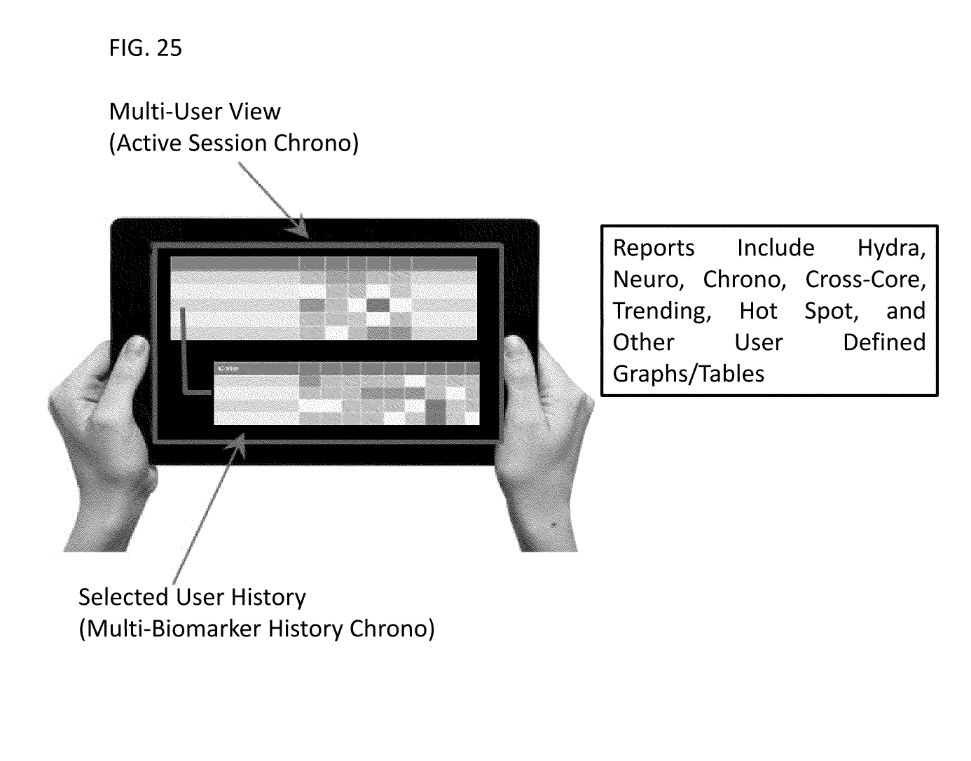

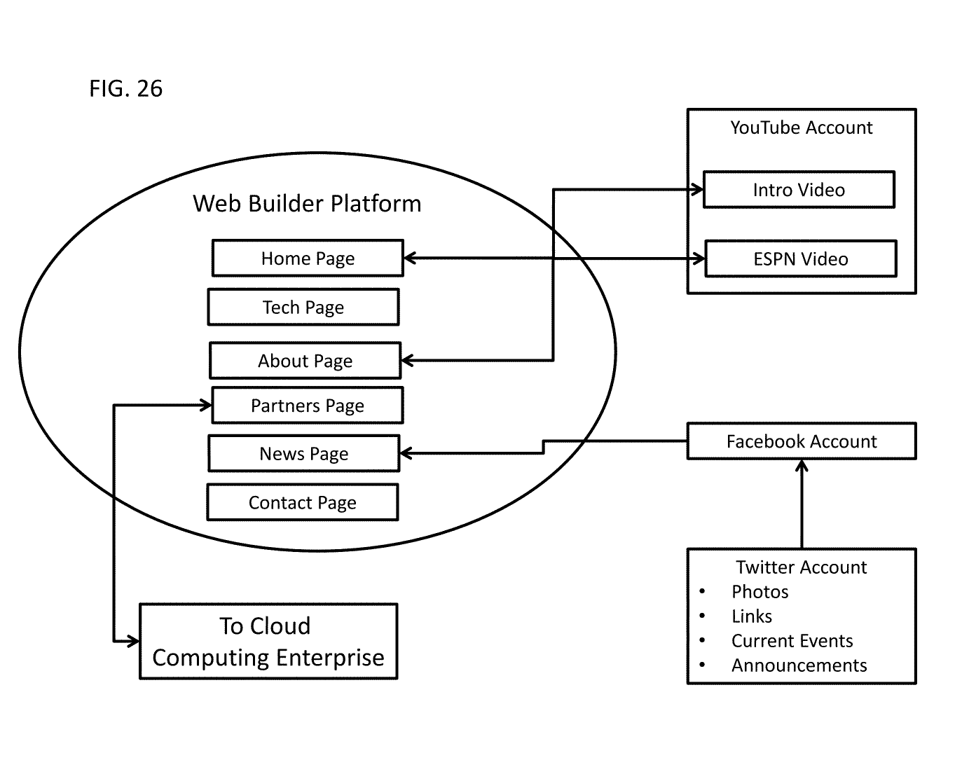

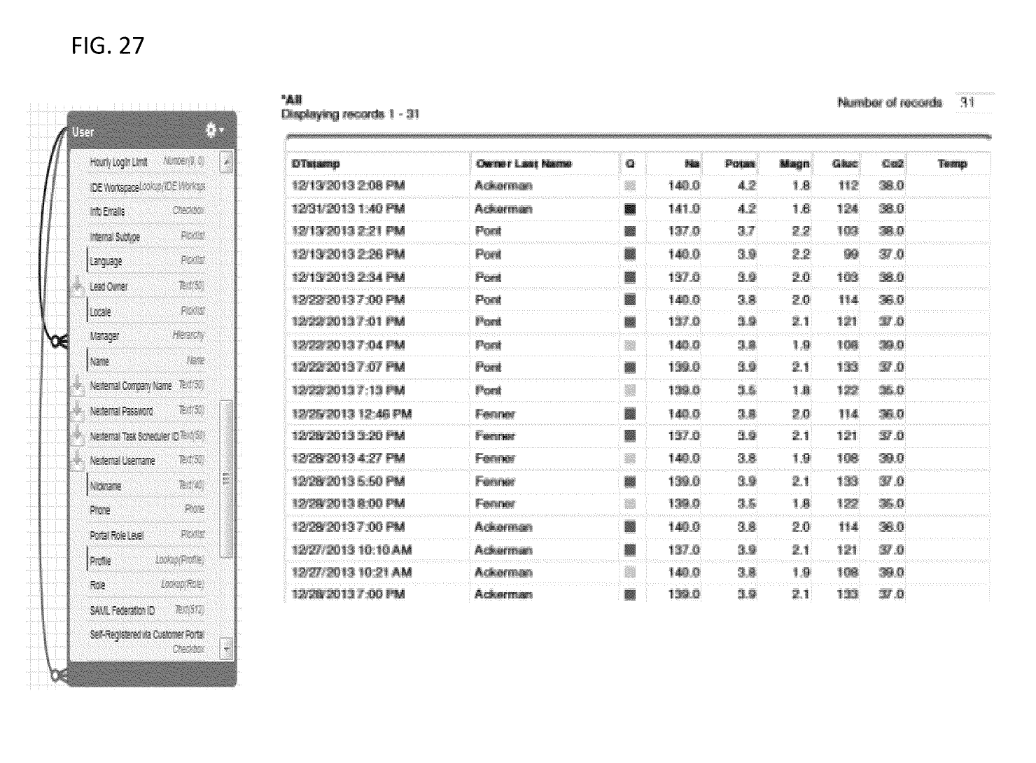

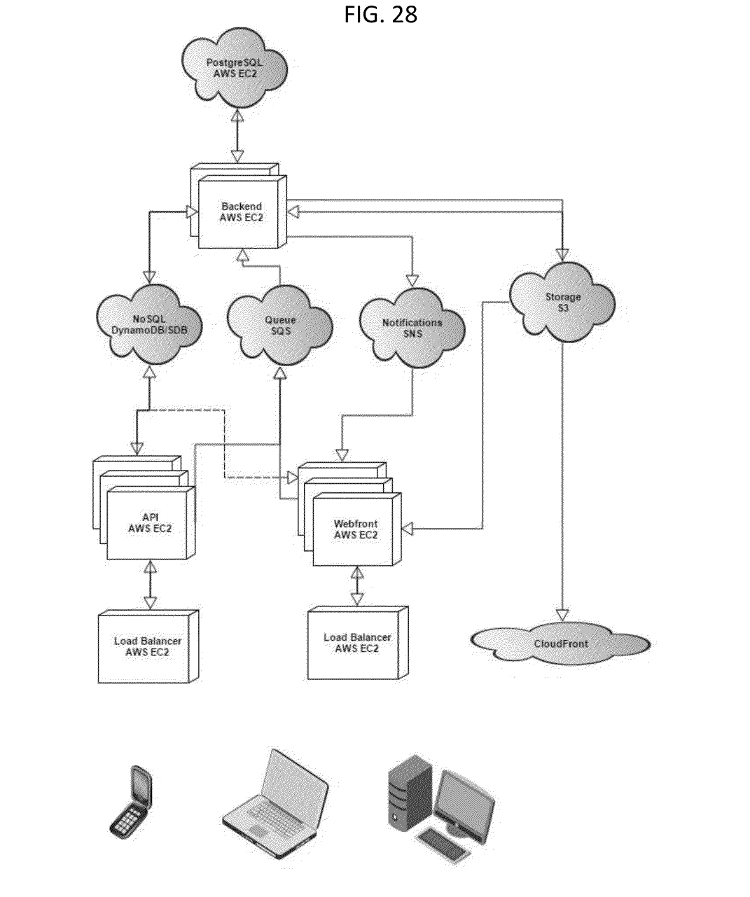

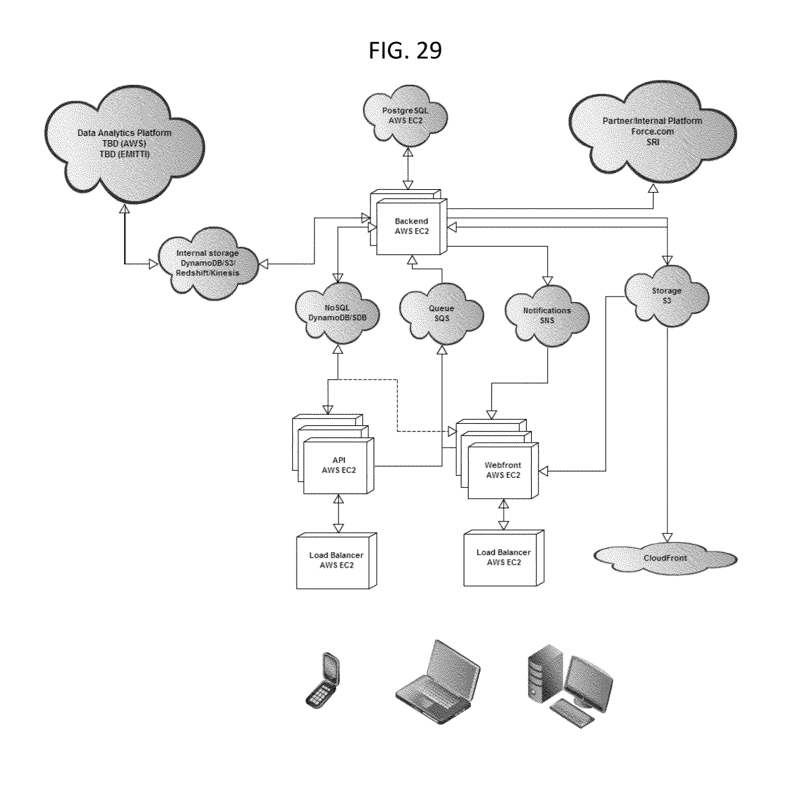

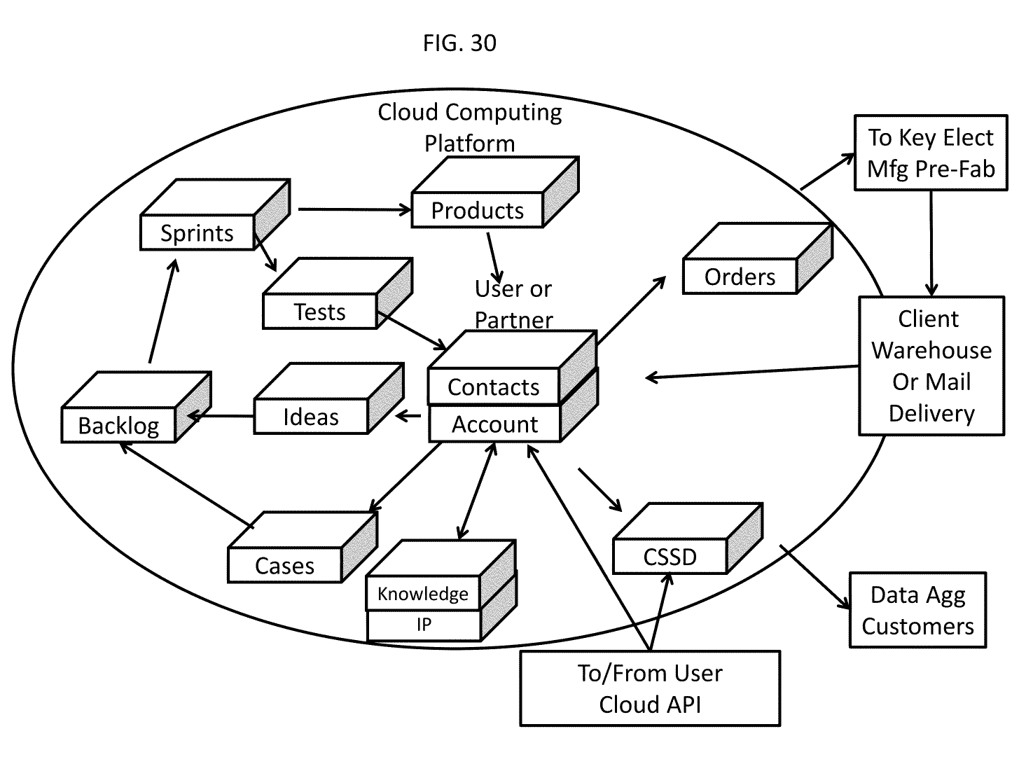

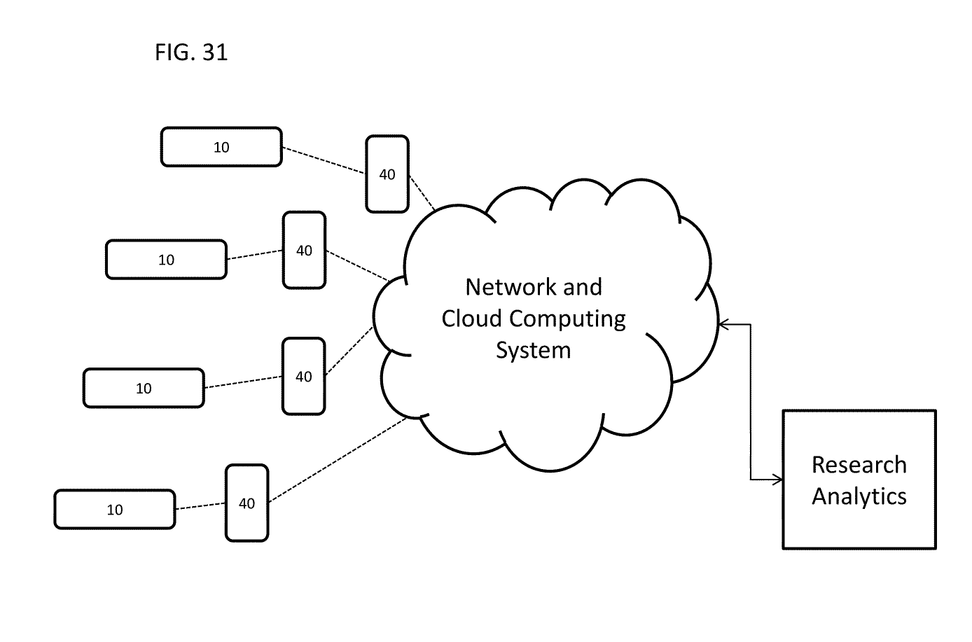

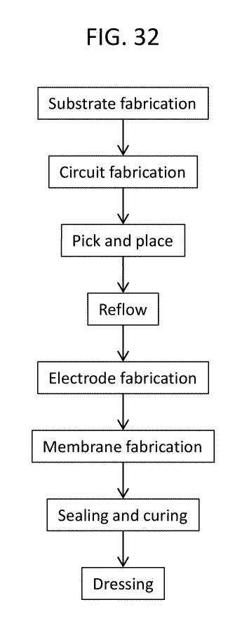

A second source of input data is the remote transceiver device. By two-way communication, the transceiver device may transmit user data such as user characteristics (including, by way of example and not limitation, height and weight, gender, fitness or conditioning level, age, transepidermal water loss (evaporated sweat), and metabolic disorder) to the microcontroller of the apparatus, which is part of an inter-integrated circuit, thus creating a “smart sensor” uniquely required for demanding use cases such as elite athletes, military, responder, and other similar users in physically demanding operations, as shown in FIGS. 17 and 18. The data to be transmitted will have been manually or automatically input in the transceiver device. For example and not limitation, as shown in FIG. 10, types of manually input data of the one or multiple users may include anthropomorphic data of the one or multiple users, such as height and weight, gender, fitness or conditioning level, age, transepidermal water loss (evaporated sweat), and metabolic disorder. The anthropomorphic data is preferably used to estimate user body surface area, which is a critical variable for accurately determining sweat loss and electrolyte loss. More preferably, estimates are a product of anthropomorphic data, gender, and age. Prior art assumes a body surface area of about 2 m² to calculate sweat loss and electrolyte loss. Using anthropomorphic variables, as in the present invention, consistently decreases calculated error rate from between about 50 and about 70 percent to less than about 10 percent, preferably. The accuracy resulting from body mass estimation revealed that persons with larger body mass, such as males, more readily adapt to physical exertion by sweating more quickly, a larger volume, and lower electrolyte concentration. Similarly, a physically fit person with a small body mass, such as a female, adapts to physical exertion by adjusting sweat flow rates and electrolyte levels. Although prior art has validly analyzed sodium, potassium, sweat rates, etc., it has failed to account for body surface area, VO2 max, and mass, thus inflating calculated error rate. These data support that sweat flow rates and electrolyte loss is strongly correlated with body size and surface area and conditioning level, which further supports the need for proper estimation of body size, such as through anthropomorphic variables. The metabolic disorder data may include diabetes. Since Type 1 diabetes is associated with reduced eccrine gland activation and, thus, lower sweat rates, the present invention may reveal user metabolic disorder. Further, Types of automatically input data may include user skin temperature, outdoor or indoor temperature and/or humidity and altitude. The automatically input data may be generated in the remote transceiver device by integrated applications, such as GPS or weather. Collectively, the data is constantly collected for up to 180 days, preferably. Alternatively, the data is constantly collected for up to 90 days or up to 120 days. Of the constantly collected data, the most recent is preferably weighted, so that the most recent physiological adaptations significantly contribute more to analyses. Together, the data transmitted to the microcontroller from the remote transceiver device represent modifying variables.

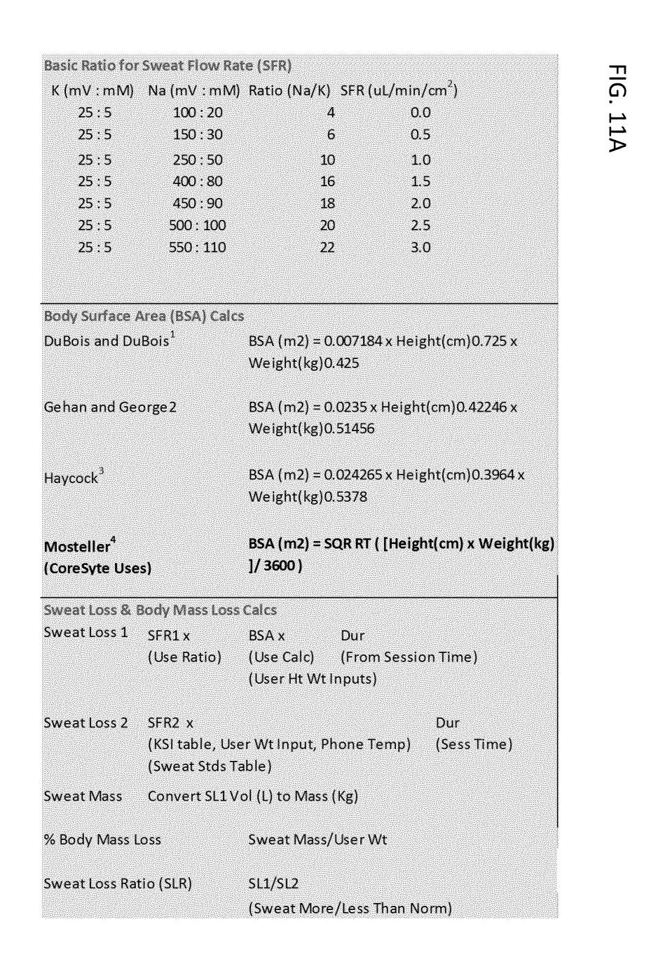

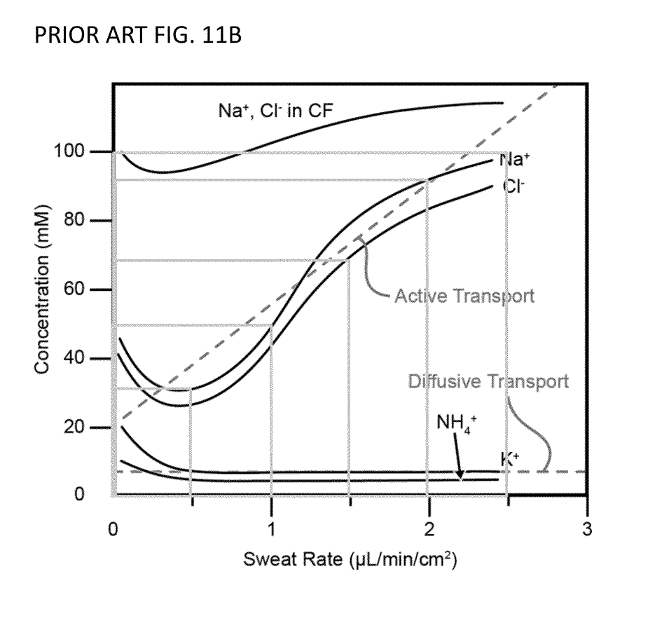

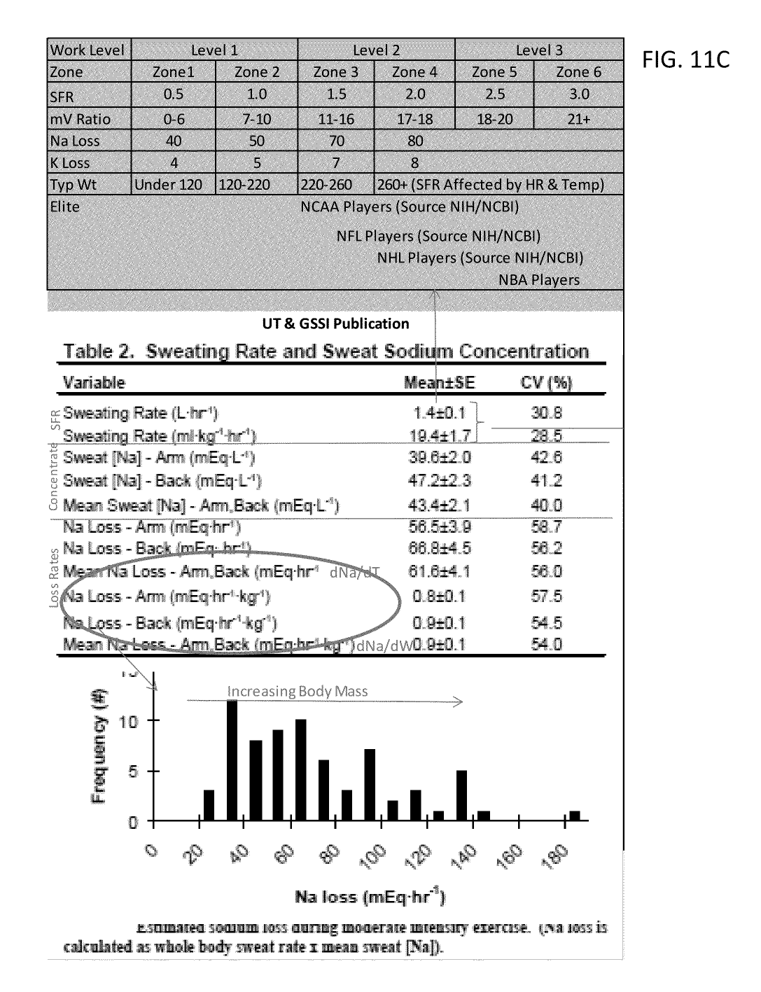

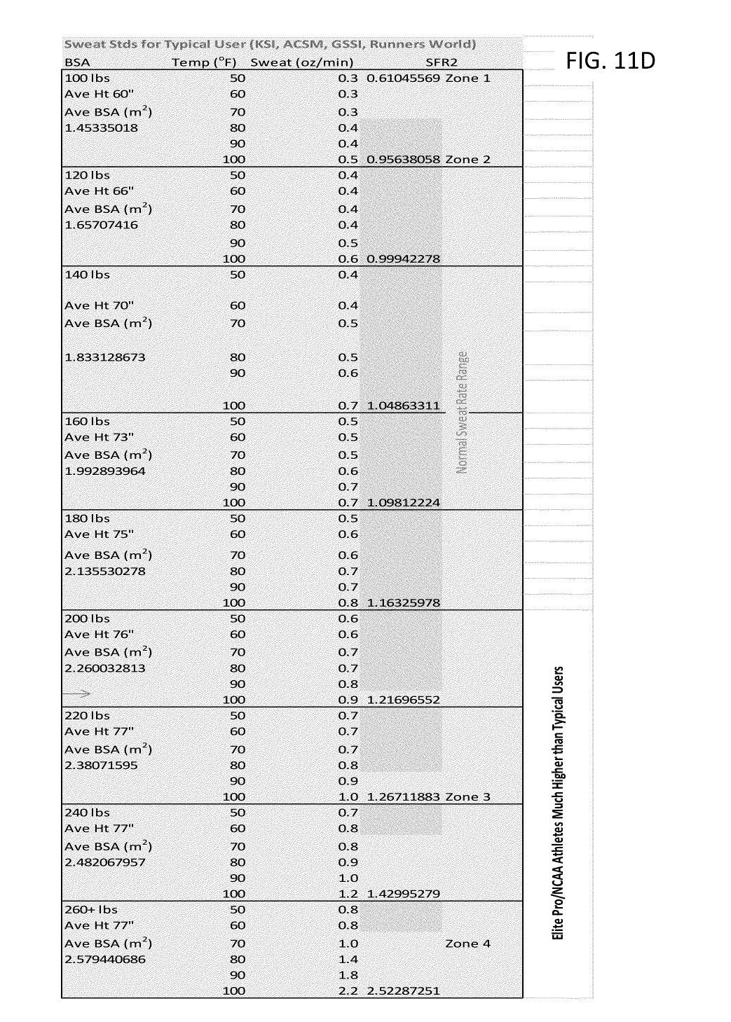

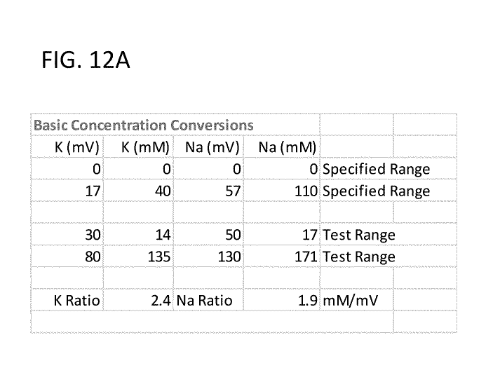

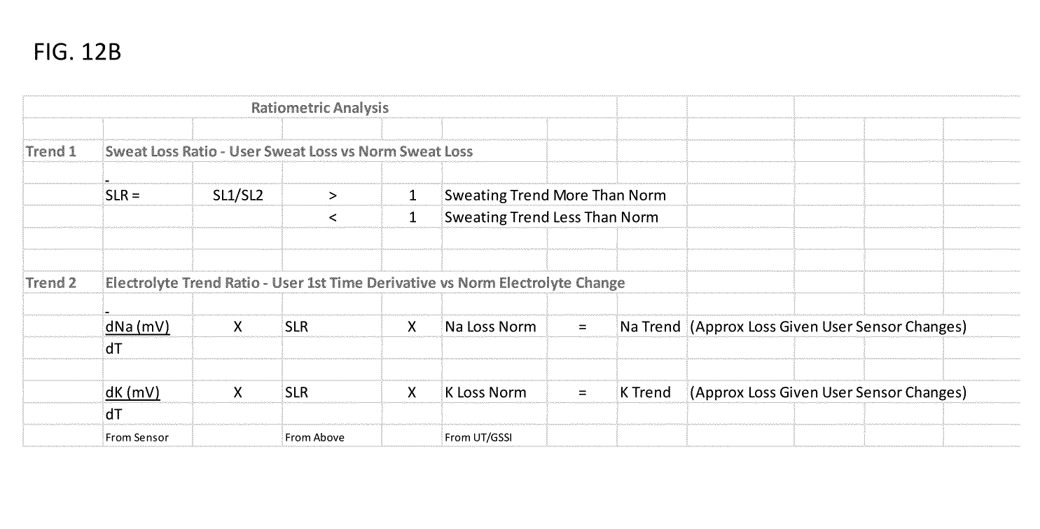

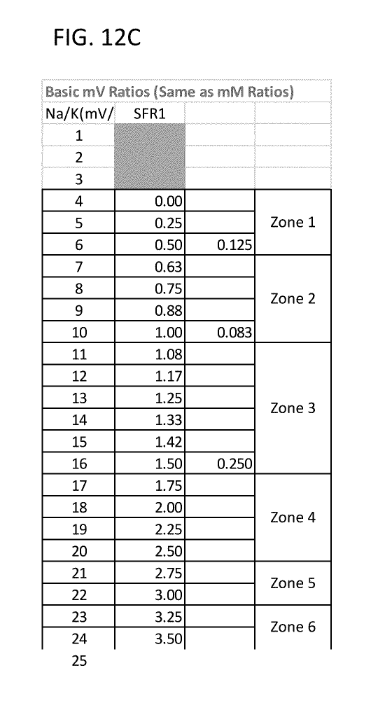

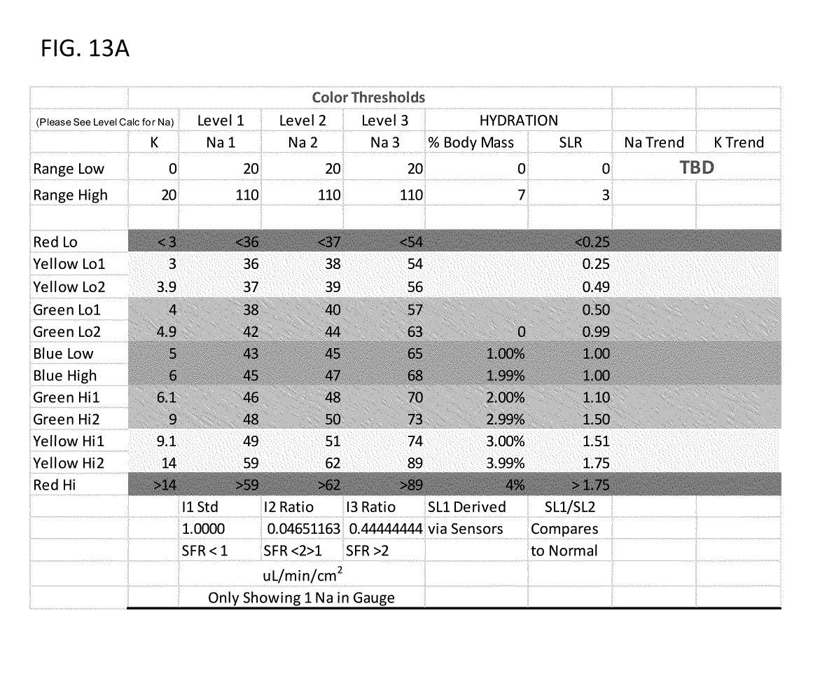

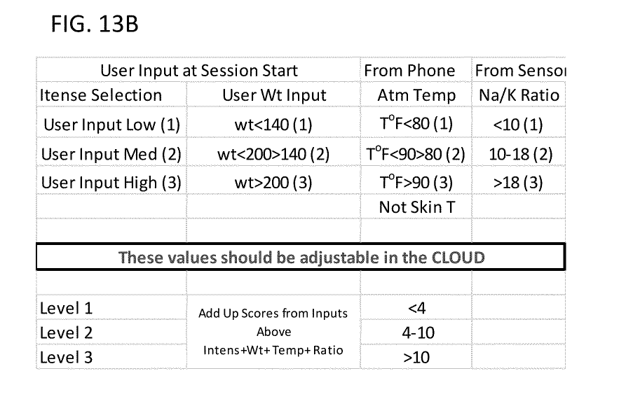

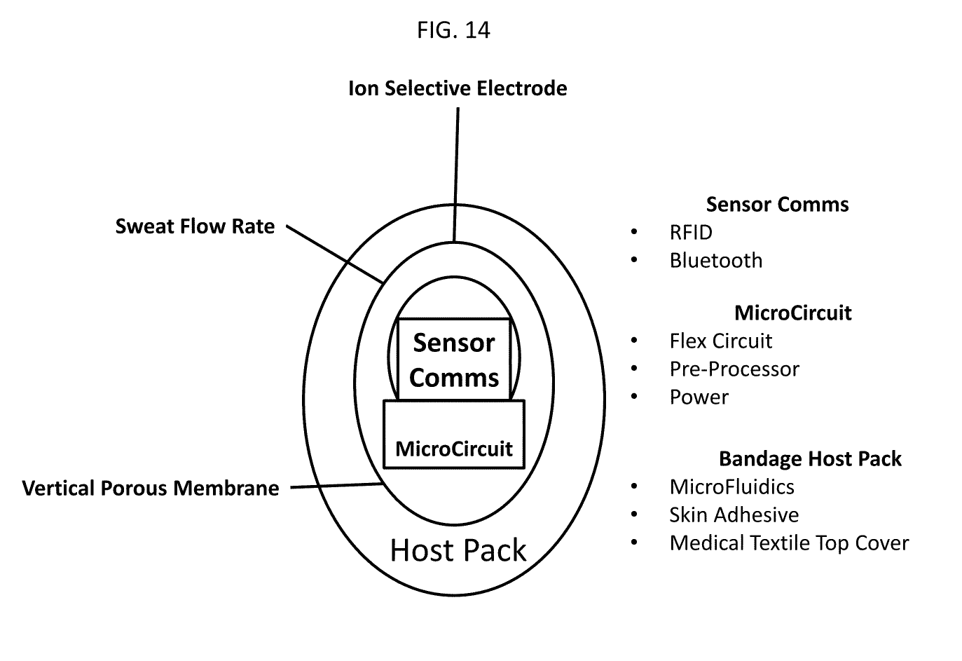

The microcontroller converts the voltage data from the biological fluid into a concentration or ratio value of the biomarker using at least one programmed algorithm. For example, as shown in FIGS. 11A-D, if the algorithm was converting the amount of sodium ions detected at the sensor into a sodium concentration, the algorithm would apply around 0.242 mM per mV of sodium. For potassium conversion, the ratio would be around 0.195 mM per mV of potassium. Embodiments for sensing sodium and potassium concentration in a liquid, including sweat, are disclosed in U.S. Pub. No. 20150057515, which is incorporated herein by reference in its entirety. In one embodiment, the sweat rate based sodium and potassium ratios of U.S. Pub. No. 20150057515 are included in algorithms of the present invention. In one embodiment, sensor data are inputs into real-time blood serum hydration, sodium concentration, and potassium concentration using absorption and extraction models that use sensor data as starting points. These calculated values are the apparatus' output data. Types of output data include but are not limited to concentrations, such as molarity, osmolarity, and osmolality, and descriptive statistics, such as averages, ratios, and trends, all of which may be categorized based on a sub-range within a larger physiological range of the biomarker, as shown in FIG. 13. The modifying variables transmitted from the remote transceiver device may modify the algorithm, which may adjust the output data.

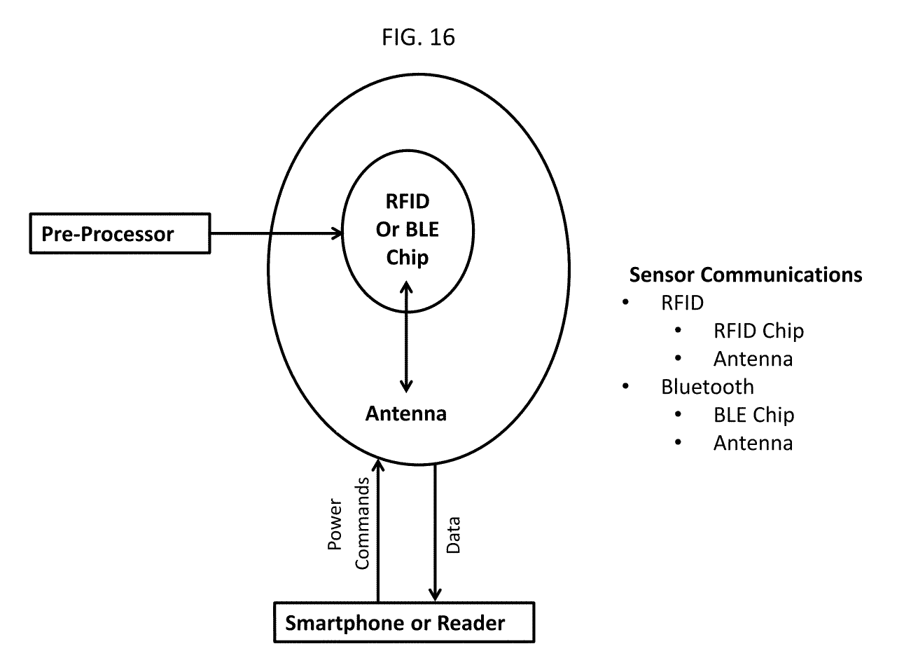

The output data is then transmitted through a wire or, preferably, through wireless communication by the transceiver antenna (including a coil) of the apparatus. The wireless transmission is provided by any suitable wireless communication, wireless network communication, standards-based or non-standards-based, by way of example and not limitation, Bluetooth, radiofrequency (including RFID), ZigBee, Wi-Fi, near field communication (NFC), or other similar commercially utilized standards. At the remote transceiver device, the output data can be viewed and assessed by the one or multiple users. The one or more users also may manipulate or further analyze the output data, such as by creating user defined graphs and tables. Preferably, the remote transceiver device is portable. More preferably, the remote transceiver device is a smartphone. Alternative devices include bulk readers, gateways, and receiver units, such as food and/or beverage dispensers with sensor and/or mobile app communication capabilities or athletic training gear including treadmills, spin bikes, ellipticals, stair climbers, and weight machines with integrated mobile communication capabilities. More alternative devices include desktop or laptop computers and tablets. FIG. 16 diagrams the communication between the sensor apparatus and the smartphone or reader, wherein power, commands, and/or data may be communicated.

From the remote transceiver device, the user may transmit processed or unprocessed data to at least one remote computer server, preferably by wireless communication, such as through a user web service. The remote computer server, which may be a network or cloud, may store the transmitted data in a library. The cloud preferably serves as a software development kit (SDK) for potential solution partners, a cloud based user app (with real time ingestion, calculation, and display), and a cloud based user store with ubiquitous access.

The preferred embodiment of the system includes an apparatus that intimately adheres to mammalian skin. The sweat from the skin is moved into the apparatus for detection of sweat biomarkers and analytes. Where on the mammal the apparatus is positioned is dependent upon, by way of example and not limitation, user preference, sweat collection patterns, or sweat production amounts at a given location.

The preferred embodiment of the apparatus is able to remain in place on mammalian skin under demanding conditions for at least 72 hours. This enables at least 8 continuous hours of extreme usage. Demanding conditions include, by way of example and not limitation, extreme temperatures such as temperatures below about −20 degrees Fahrenheit and temperatures above about 120 degrees Fahrenheit. Preferably, the device is operable to adhere to mammalian skin for at least 72 hours and operate for at least 8 continuous hours between about −10 degrees Fahrenheit and about 110 degrees Fahrenheit. In another embodiment, the temperature range is between about 0 degrees Fahrenheit and about 100 degrees Fahrenheit. Other extreme usage and demanding conditions include wet conditions. Wet conditions include natural wet conditions such as ponds, lakes, streams, rivers, and the ocean, as well as artificial wet conditions such as pools. The device of the present invention is preferably operable to adhere to mammalian skin for at least 72 hours and operate for at least 8 continuous hours when exposed to naturally occurring elements and compounds in seawater, including but not limited to oxygen, hydrogen, sodium, chlorine, magnesium, vanadium, sulfur, calcium, potassium, bromine, and carbon, without affecting the readings of the device. The device is also preferably operable for pH levels between about 7 and about 8. In another embodiment, the pH levels are between about 7.2 and 7.8. In a preferred embodiment, the pH levels are between about 7.2 and about 7.6. Preferably, the device is operable in environments up to about 40% salinity. In other embodiments, the device is operable in environments up to about 30% salinity, 20% salinity, and 10% salinity. The device is also operable to adhere to mammalian skin for at least 72 hours and operate for at least 8 continuous hours when continuously exposed to high levels of pool chemicals, including but not limited to chlorine, calcium hypochlorite, sodium hypochlorite, hypochlorous acid, and bromide. The device is also preferably operable to operable to adhere to mammalian skin for at least 72 hours and operate for at least 8 continuous hours under pressure due to submersion. By way of example and not limitation, depths of up to about 400 feet (water pressure of about 173.4 psi) can be tolerated. In another embodiment, depths of up to about 300 feet (water pressure of about 130.1 psi) can be tolerated. In yet another embodiment, depths of up to about 200 feet (water pressure of about 86.7 psi) can be tolerated. In another embodiment, depths of up to about 100 feet (water pressure of about 43.3 psi) can be tolerated. In another embodiment, depths of up to about 50 feet (water pressure of about 21.7 psi) can be tolerated. Preferably the device is operable to adhere to mammalian skin for at least 72 hours and operate for at least 8 continuous hours for any depth when adhered to a mammal in a deep sea exploration machine. The device is also operable to adhere to mammalian skin for at least 72 hours and operate for at least 8 continuous hours when used in conjunction with scuba diving wet suits and equipment. The device is also operable to adhere to mammalian skin for at least 72 hours and operate for at least 8 continuous hours when attached to mammalian skin at high altitudes. Preferably, the device is operable for altitudes up to about 30,000 feet.

The apparatus is operable to determine a measured amount of transepidermal sweat and/or a measured amount of evaporative sweat and an estimated amount of transepidermal sweat and/or an estimated amount of evaporative sweat. In one embodiment, estimated amounts are determined based on body surface area, mass, gender, fitness level, weight, and/or age. In another embodiment, the apparatus is operable to compare the estimated amount of transepidermal sweat and/or the estimated amount of evaporative sweat to the measured amount of transepidermal sweat and/or the measured amount of evaporative sweat and provide a status based on the comparison of the estimated amount of transepidermal sweat and/or the estimate amount of evaporative sweat to the measured amount of transepidermal sweat and/or the measured amount of evaporative sweat. In another embodiment, the apparatus uses a tangible/quantifiable fitness level in combination with sweat biomarker ratios in order to calculate real-time sweat rates. In another embodiment, the apparatus uses gender factors in order to improve sweat flow rate accuracy. In another embodiment, the apparatus uses consumption refresh models, exact custom formula to return to start condition.

The apparatus is also operable to model losses and consumption of sweat in order to estimate blood serum characteristics at a time before use, at the start of use, in real-time, or at a time after use. In one embodiment, the apparatus is operable to predict performance erosion and injury probability based on the analysis of at least one biological fluid biomarker. In another embodiment, the apparatus is operable to use the analysis of sweat to provide corrective action recommendations.

Exemplary technologies regarding sensor technology, analysis, communication, and manufacture are illustrated by the following: PCT patent application publication no. PCT/US2013/035092 for sweat stimulation, collection and sensing systems by Heikenfeld, et al. filed Apr. 3, 2013; PCT patent application publication no. PCT/US2014/061083 for devices for integrated, repeated, prolonged, and/or reliable sweat stimulation and biosensing by Heikenfeld and Sonner filed Oct. 17, 2014; PCT patent application publication no. PCT/US2014/061098 for sweat sensing with chronological assurance by Heikenfeld filed Oct. 17, 2014; U.S. provisional patent application Ser. No. 62/003,675 for advanced sweat sensor adhesion, hermetic, and fluidic strategies by Heikenfeld filed May 28, 2014; U.S. provisional patent application Ser. No. 62/003,692 for dermal transport and biomarker sensing devices with preferential targeting of sweat ducts by Heikenfeld filed MAy 28, 2014; U.S. provisional patent application Ser. No. 62/003,707 for device construction and methods for prolonged and reliable stimulation and sensing by Heikenfeld filed May 28, 2014; U.S. provisional patent application Ser. No. 62/003,715 for vertical flow porous electrode membrane devices for sensing by Heikenfeld and Sonner filed May 28, 2014; U.S. provisional patent application Ser. No. 62/003,233 for combinatorial sensing of sweat biomarkers using simple potentiometry and impedance measurements by Heikenfeld, et al. filed Jul. 11, 2014; U.S. provisional patent application Ser. No. 62/053,388 for sweat sensing with analytical assurance by Heikenfeld filed Sep. 22, 2014; U.S. provisional patent application Ser. No. 62/064,009 for patch communication security and compliance by Heikenfeld, et al. filed Oct. 15, 2014; U.S. provisional patent application Ser. No. 62/074,295 for advanced adhesives for chronological sweat sensors by Heikenfeld filed Nov. 3, 2014; and U.S. provisional patent application Ser. No. 62/114,835 for devices with reduced sweat volumes between sensors and sweat glands by Heikenfeld filed Feb. 11, 2015; each of which is incorporated herein by reference in its entirety.

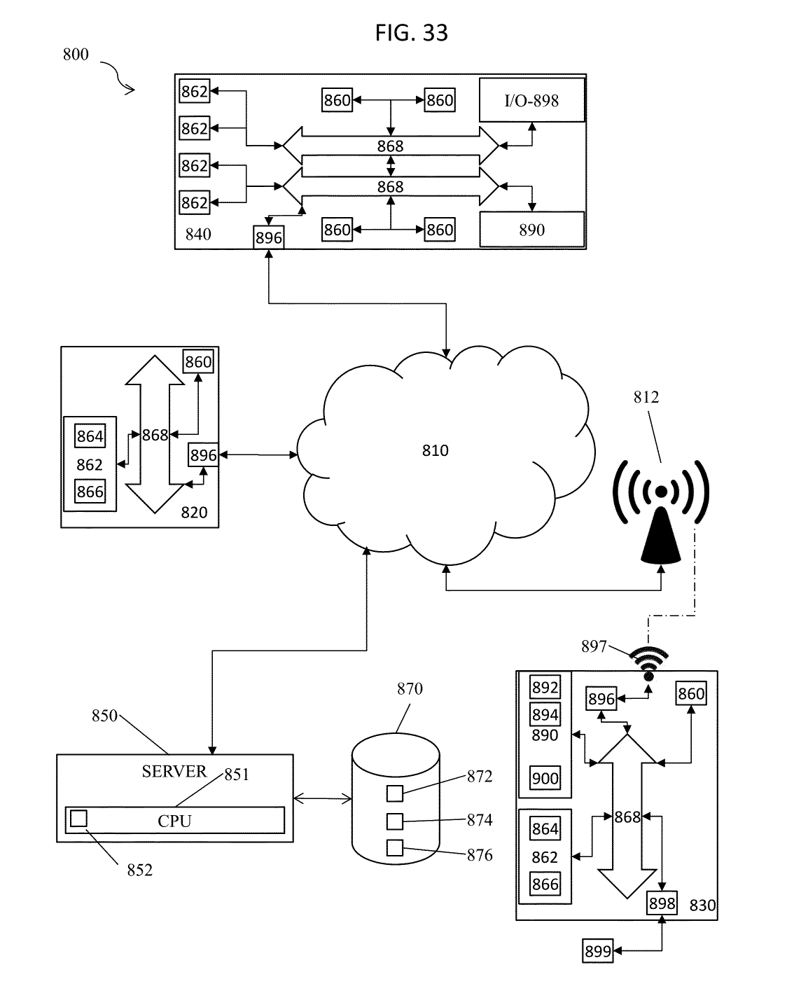

FIG. 33 is a schematic diagram of an embodiment of the invention illustrating a cloud-based computer system, generally described as 800, having a network 810, a plurality of computing devices 820, 830, 840, a server 850 and a database 870. The server 850 is constructed, configured and coupled to enable communication over a network 810 with a computing devices 820, 830, 840. The server 850 includes a processing unit 851 with an operating system 852. The operating system 852 enables the server 850 to communicate through network 810 with the remote, distributed user devices. Database 870 may house an operating system 872, memory 874, and programs 876.

In one embodiment of the invention, the system 800 includes a cloud-based network 810 for distributed communication via a wireless communication antenna 812 and processing by a plurality of mobile communication computing devices 830. In another embodiment of the invention, the system 800 is a virtualized computing system capable of executing any or all aspects of software and/or application components presented herein on the computing devices 820, 830, 840. In certain aspects, the computer system 800 may be implemented using hardware or a combination of software and hardware, either in a dedicated computing device, or integrated into another entity, or distributed across multiple entities or computing devices.

By way of example, and not limitation, the computing devices 820, 830, 840 are intended to represent various forms of digital computers 820, 840, 850 and mobile devices 830, such as a server, blade server, mainframe, mobile phone, a personal digital assistant (PDA), a smart phone, a desktop computer, a netbook computer, a tablet computer, a workstation, a laptop, and other similar computing devices. The components shown here, their connections and relationships, and their functions, are meant to be exemplary only, and are not meant to limit implementations of the invention described and/or claimed in this document

In one embodiment, the computing device 820 includes components such as a processor 860, a system memory 862 having a random access memory (RAM) 864 and a read-only memory (ROM) 866, and a system bus 868 that couples the memory 862 to the processor 860. In another embodiment, the computing device 830 may additionally include components such as a storage device 890 for storing the operating system 892 and one or more application programs 894, a network interface unit 896, and/or an input/output controller 898. Each of the components may be coupled to each other through at least one bus 868. The input/output controller 898 may receive and process input from, or provide output to, a number of other devices 899, including, but not limited to, alphanumeric input devices, mice, electronic styluses, display units, touch screens, signal generation devices (e.g., speakers) or printers.

By way of example, and not limitation, the processor 860 may be a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated or transistor logic, discrete hardware components, or any other suitable entity or combinations thereof that can perform calculations, process instructions for execution, and/or other manipulations of information.

In another implementation, shown as 840 in FIG. 33, multiple processors 860 and/or multiple buses 868 may be used, as appropriate, along with multiple memories 862 of multiple types (e.g., a combination of a DSP and a microprocessor, a plurality of microprocessors, one or more microprocessors in conjunction with a DSP core).

Also, multiple computing devices may be connected, with each device providing portions of the necessary operations (e.g., a server bank, a group of blade servers, or a multi-processor system). Alternatively, some steps or methods may be performed by circuitry that is specific to a given function.

According to various embodiments, the computer system 800 may operate in a networked environment using logical connections to local and/or remote computing devices 820, 830, 840, 850 through a network 810. A computing device 830 may connect to a network 810 through a network interface unit 896 connected to the bus 868. Computing devices may communicate communication media through wired networks, direct-wired connections or wirelessly such as acoustic, RF or infrared through an antenna 897 in communication with the network antenna 812 and the network interface unit 896 , which may include digital signal processing circuitry when necessary. The network interface unit 896 may provide for communications under various modes or protocols.

In one or more exemplary aspects, the instructions may be implemented in hardware, software, firmware, or any combinations thereof. A computer readable medium may provide volatile or non-volatile storage for one or more sets of instructions, such as operating systems, data structures, program modules, applications or other data embodying any one or more of the methodologies or functions described herein. The computer readable medium may include the memory 862, the processor 860, and/or the storage media 890 and may be a single medium or multiple media (e.g., a centralized or distributed computer system) that store the one or more sets of instructions 900. Non-transitory computer readable media includes all computer readable media, with the sole exception being a transitory, propagating signal per se. The instructions 900 may further be transmitted or received over the network 810 via the network interface unit 896 as communication media, which may include a modulated data signal such as a carrier wave or other transport mechanism and includes any delivery media. The term “modulated data signal” means a signal that has one or more of its characteristics changed or set in a manner as to encode information in the signal.

Storage devices 890 and memory 862 include, but are not limited to, volatile and non-volatile media such as cache, RAM, ROM, EPROM, EEPROM, FLASH memory or other solid state memory technology, disks or discs (e.g., digital versatile disks (DVD), HD-DVD, BLU-RAY, compact disc (CD), CD-ROM, floppy disc) or other optical storage, magnetic cassettes, magnetic tape, magnetic disk storage or other magnetic storage devices, or any other medium that can be used to store the computer readable instructions and which can be accessed by the computer system 800.

It is also contemplated that the computer system 800 may not include all of the components shown in FIG. 33, may include other components that are not explicitly shown in FIG. 33, or may utilize an architecture completely different than that shown in FIG. 33. The various illustrative logical blocks, modules, elements, circuits, and algorithms described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application (e.g., arranged in a different order or partitioned in a different way), but such implementation decisions should not be interpreted as causing a departure from the scope of the present invention.

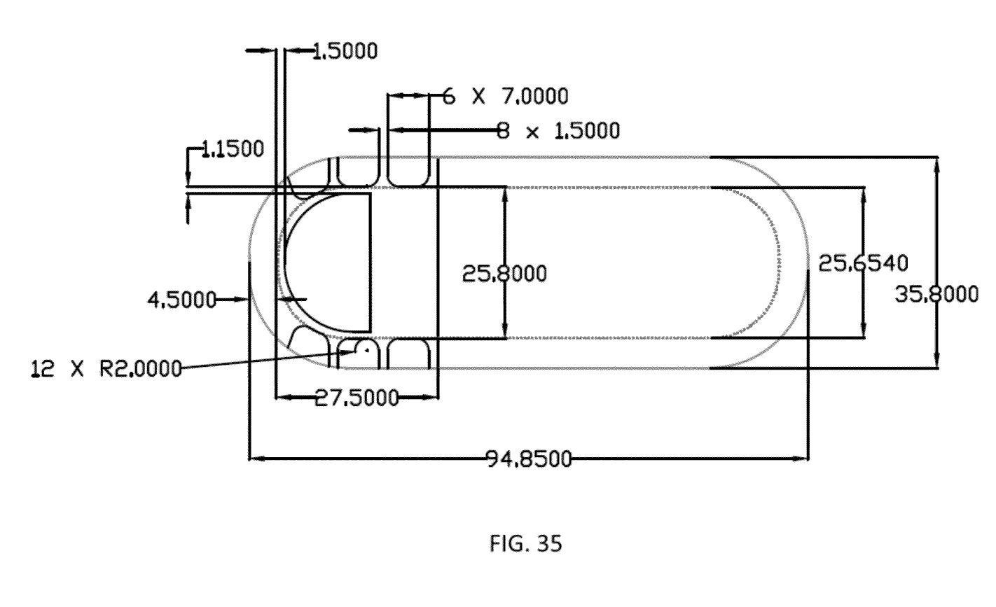

FIG. 35 shows a top perspective view of one embodiment of the sensor apparatus. The flex circuit is substantially centrally located on the bandage material and adhesive. Wicking paper is utilized to move bodily fluid, particularly sweat, through the sensor apparatus. The reverse “D” shaped hole is through the adhesive and the wicking paper. All dimensions in FIG. 35 are in MM or VOS, as appropriate.

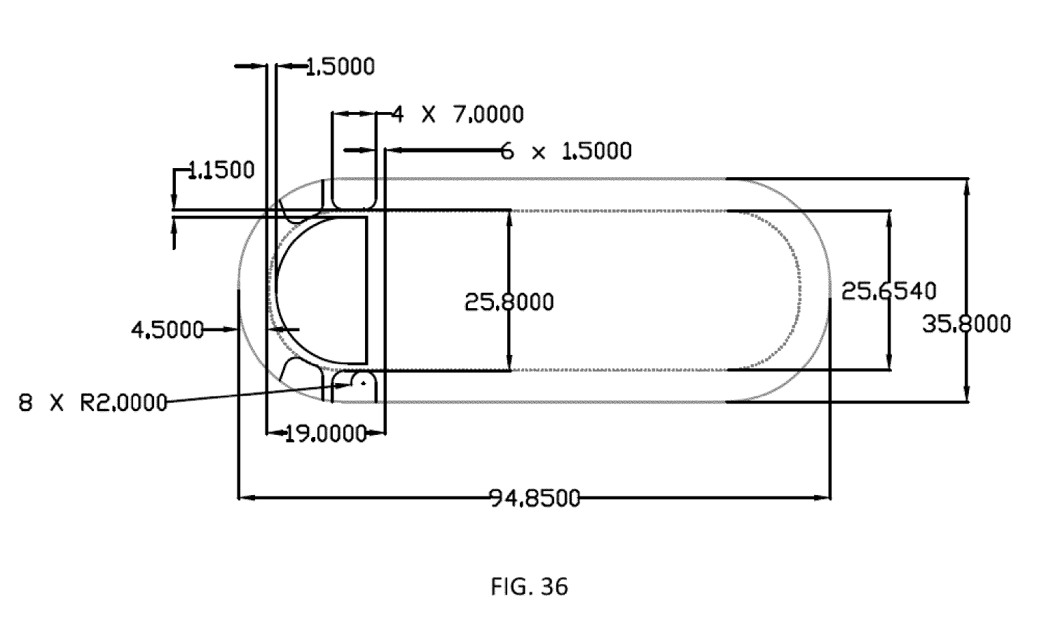

FIG. 36 shows a top perspective view of another embodiment of the sensor apparatus. The flex circuit is substantially centrally located on the bandage material and adhesive. Wicking paper is utilized to move bodily fluid, particularly sweat, through the sensor apparatus. The reverse “D” shaped hole is through the adhesive and the wicking paper. All dimensions in FIG. 36 are in MM or VOS, as appropriate.

Certain modifications and improvements will occur to those skilled in the art upon a reading of the foregoing description, by way of example, a device having at least one microprocessor for storing data may be operable in the device before data transmission. Another example includes other advanced sensors, as well as being incorporated into smart fabrics and protective wear. Advanced sensors include advanced sweat biomarkers, pulse rate breath rate, micro EKG, micro O2, picture log, voice log, voice translate, tissue safe X-ray and combinations thereof. Smart fabrics incorporate the present invention and include active heat/cooling, kinetic energy generation, electromagnetic energy harvesting, wearable energy storage, wearable data storage, wearable processing, wearable communications, elastomeric actuators, and combinations thereof. More generally, the apparatus may be part of apparel and material for lower body clothing and upper body clothing, as well as protective wear such as helmets, gloves, and footwear. The present invention is also incorporated into enhanced protective wear such as enhanced helmets, gloves and footwear. Enhanced helmets include those with Mill, 3D audio, visual enhancement, mixed reality, breath sensors, aerosol nutrition and combinations thereof. Enhanced gloves include touch communications, elastomeric grip, gesture control and combinations thereof. Enhanced footwear includes power generation boots, 3D tracking, tactile alerts, communication transceivers, and combinations thereof.

By way of definition and description supporting the claimed subject matter, preferably, the present invention includes communication methodologies for transmitting data, data packets, messages or messaging via a communication layer. Wireless communications over a network are preferred. Correspondingly, and consistent with the communication methodologies for transmitting data or messaging according to the present invention, as used throughout this specification, figures and claims, wireless communication is provided by any reasonable protocol or approach, by way of example and not limitation, Bluetooth, Wi-Fi, cellular, ZigBee, near field communication (NFC), radiofrequency (including RFID), or other similar commercially utilized standards; the term “ZigBee” refers to any wireless communication protocol adopted by the Institute of Electronics & Electrical Engineers (IEEE) according to standard 802.15.4 or any successor standard(s), the term “Wi-Fi” refers to any communication protocol adopted by the IEEE under standard 802.11 or any successor standard(s), the term “WiMax” refers to any communication protocol adopted by the IEEE under standard 802.16 or any successor standard(s), and the term “Bluetooth” refers to any short-range communication protocol implementing IEEE standard 802.15.1 or any successor standard(s). Additionally or alternatively to WiMax, other communications protocols may be used, including but not limited to a “1G” wireless protocol such as analog wireless transmission, first generation standards based (IEEE, ITU or other recognized world communications standard), a “2G” standards based protocol such as “EDGE or CDMA 2000 also known as 1× RTT”, a 3G based standard such as “High Speed Packet Access (HSPA) or Evolution for Data Only (EVDO), any accepted 4G standard such as “IEEE, ITU standards that include WiMax, Long Term Evolution “LTE” and its derivative standards, any Ethernet solution wireless or wired, or any proprietary wireless or power line carrier standards that communicate to a client device or any controllable device that sends and receives an IP based message. The term “High Speed Packet Data Access (HSPA)” refers to any communication protocol adopted by the International Telecommunication Union (ITU) or another mobile telecommunications standards body referring to the evolution of the Global System for Mobile Communications (GSM) standard beyond its third generation Universal Mobile Telecommunications System (UMTS) protocols. The term “Long Term Evolution (LTE)” refers to any communication protocol adopted by the ITU or another mobile telecommunications standards body referring to the evolution of GSM-based networks to voice, video and data standards anticipated to be replacement protocols for HSPA. The term “Code Division Multiple Access (CDMA) Evolution Date-Optimized (EVDO) Revision A (CDMA EVDO Rev. A)” refers to the communication protocol adopted by the ITU under standard number TIA-856 Rev. A.

It will be appreciated that embodiments of the invention described herein may be comprised of one or more conventional processors and unique stored program instructions that control the one or more processors to implement, in conjunction with certain non-processor circuits, some, most, or all of the functions for the systems and methods as described herein. The non-processor circuits may include, but are not limited to, radio receivers, radio transmitters, antennas, modems, signal drivers, clock circuits, power source circuits, relays, current sensors, and user input devices. As such, these functions may be interpreted as steps of a method to distribute information and control signals between devices. Alternatively, some or all functions could be implemented by a state machine that has no stored program instructions, or in one or more application specific integrated circuits (ASICs), in which each function or some combinations of functions are implemented as custom logic. Of course, a combination of the two approaches could be used. Thus, methods and means for these functions have been described herein. Further, it is expected that one of ordinary skill in the art, notwithstanding possibly significant effort and many design choices motivated by, for example, available time, current technology, and economic considerations, when guided by the concepts and principles disclosed herein, will be readily capable of generating such software instructions, programs and integrated circuits (ICs), and appropriately arranging and functionally integrating such non-processor circuits, without undue experimentation.

The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

What is claimed is:

1. A device for analyzing biological fluid and transmitting and storing biological fluid data for extended wear under demanding conditions comprising:
a fluid sensor apparatus for sensing and analyzing at least one biological fluid, wherein the fluid sensor apparatus is multi-layered, including:
a macrofluidic, double-sided adhesive layer;
an electronic layer comprising at least one electrochemical sensor, a microcontroller, and a transceiver antenna coil;
a microfluidic management layer; and
a vapor porous, top protective layer;
wherein the macrofluidic, double-sided adhesive layer is removably adhered to a surface;
wherein the electronic layer is intimately adhered to the macrofluidic, double-sided adhesive layer;
wherein the microfluidic management layer circumferentially surrounds the at least one electrochemical sensor of the electronic layer;
wherein the vapor porous, top protective layer is placed on and completely covers the microfluidic management layer and electronic layer; and
wherein the vapor porous, top protective layer is adhered to the macrofluidic, double-sided adhesive layer.

2. The device of claim 1, wherein the fluid sensor apparatus is flexible.

3. The device of claim 1, wherein the surface that the macrofluidic, double-sided adhesive layer is removably adhered to is skin.

4. The device of claim 3, wherein the skin is mammalian skin.

5. The device of claim 1, wherein the biological fluid is sweat.

6. The device of claim 1, wherein the at least one electrochemical sensor of the electronic layer is operable to detect and continuously analyze at least one biomarker of the biological fluid.

7. The device of claim 6, wherein the at least one biomarker of the biological fluid includes electrolytes, small molecules, proteins, and/or metabolites.

8. The device of claim 1, wherein the microcontroller receives user data to customize rates at which the at least one biological fluid is sensed and analyzed, to toggle circuit power of the microcontroller, communicate via multiple channels and/or buses with a computing device biological fluid data via multiple protocols concurrently in real-time to a computing device with software operable thereon for receiving, storing, and analyzing the biological fluid data, and define data collection along with transmission characteristics in real time for optimization of power and the electrochemical sensor features for a user, wherein the user data includes height, weight, gender, fitness level, conditioning level, age, transepidermal water loss, metabolic disorder, environmental temperature, environmental pressure, environmental sodium concentration, environmental chlorine concentration, and/or combinations thereof.

9. The device of claim 1, wherein the device wirelessly transmits the sensed and stored biological fluid data via multiple protocols concurrently in real-time to a computing device with software operable thereon for receiving, storing, and analyzing the biological fluid data.

10. The device of claim 8, wherein the computing device calculates biological fluid concentration ratios of the biological fluid and provides visual representations of the received and analyzed data on a graphic user interface of the computing device.

11. The device of claim 1, wherein the fluid sensor apparatus further includes gold probes or electrodes.

12. A device for analyzing biological fluid and transmitting and storing biological fluid data for elite human performers in demanding conditions comprising:
a fluid sensor apparatus for sensing and analyzing at least one biological fluid, wherein the fluid sensor apparatus includes:
a skin adhesive layer including at least one laser cut, macrofluidic pore; and
an electronic layer including at least one electrochemical sensor unit that includes at least one electrode;
wherein the skin adhesive layer is operable to allow the biological fluid to flow through the at least one macrofluidic pore; and
wherein the at least one electrode of the at least one electrochemical sensor unit is operable to detect at least one biomarker of the biological fluid when the biological fluid comes into contact with the at least one electrode.

13. The device of claim 12, wherein the fluid sensor apparatus further includes a woven textile protective top layer, wherein the woven textile protective top layer facilitates evaporation of the biological fluid after the biological fluid contacts the at least one electrode of the at least one electrochemical sensor unit.

14. The device of claim 12, wherein the biological fluid is sweat.

15. The device of claim 12, wherein the fluid sensor apparatus further includes a microcontroller that is operable to receive input data from at least one portable device.

16. The device of claim 15, wherein the input data received by the microcontroller comprises information about one or multiple users including height, weight, gender, fitness or conditioning level, age, trans epidermal weight loss, and/or metabolic disorder.

17. The device of claim 12, wherein the at least one electrochemical sensor unit is further operable to measure characteristics of the at least one biomarker including concentration, molarity, osmolarity, and/or osmolality.

18. A device for analyzing biological fluid and transmitting and storing biological fluid data comprising:
a fluid sensor apparatus for sensing and analyzing at least one biological fluid;
wherein the fluid sensor apparatus includes an electronic layer including at least one electrochemical sensor unit that includes at least one electrode; and
wherein the at least one electrode includes an ionophore polymer coating.

19. The device of claim 18, wherein the ionophore polymer coating is approximately 2 microliters and does not extend more than 0.5 millimeters from the edge of the at least one electrode such that the ionophore polymer coating remains in place for at least 72 hours and provides for at least 8 hours of continuous use under demanding conditions, wherein the demanding conditions include pressure of about 21.7 psi and/or temperatures ranging from about 0 degrees Fahrenheit to about 100 degrees Fahrenheit.

20. The device of claim 18, wherein the at least one electrode is operable to detect at least one biomarker of the biological fluid when the biological fluid comes into contact with the electrode.
